(19) United States
(12) Patent Application Publication
(10) Pub. No.: US 2014/0350273 A1
(43) Pub. Date: Nov. 27, 2014

(54) PEDIATRIC USES OF CABAZITAXEL

(71) Applicant: AVENTIS PHARMA S.A., Antony (FR)

(72) Inventors: Sidhu S. SUKHVINDER, L'hay-les-Roses (FR); Patricia VRIGNAUD, Combs la Ville (FR)

(21) Appl. No.: 14/454,364

(22) Filed: Aug. 7, 2014

Related U.S. Application Data
(63) Continuation of application No. PCT/EP2013/052518, filed on Feb. 8, 2013.

(30) Foreign Application Priority Data
Feb. 10, 2012 (EP) 12305151.8
Oct. 30, 2012 (EP) 12306352.1

(51) Int. Cl. A61K 31/337

(52) U.S. Cl. CPC A61K 31/337
USPC 549/510

(57) ABSTRACT

The present invention relates to the compound of formula (I):

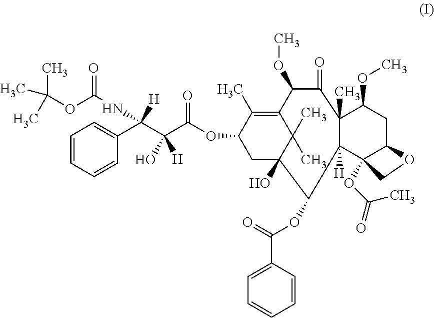 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]

which may be in the form of an anhydrous base, a hydrate or a solvate,
for its use for the treatment of pediatric cancers.

This application is a continuation of International Patent Application No. PCT/EP2013/052518, filed Feb. 8, 2013, which is incorporated herein by reference; and claims priority to European Application No. 12305151.8, filed Feb. 10, 2012 and to European Application No. 12306352.1, filed Oct. 30, 2012.

The present invention concerns new pediatric uses of cabazitaxel. It also concerns a new method for treating children and young adults.

Over the past 20 years, there has been some increase in the incidence of children diagnosed with all forms of invasive cancer. Long-term trends in incidence for leukemias and brain tumors, the most common childhood cancers, show patterns that are somewhat different from the others. Incidence of childhood leukemias appeared to rise in the early 1980s. Rates in the succeeding years have shown no consistent upward or downward trend.

While leukemia is the most common pediatric malignancy, brain tumors are the most common solid tumors, representing 21% of all cancers in children, followed by neuroblastoma (8.3%), nephroblastoma (5.9%), bone tumors (4.6%) such as Osteosarcoma, Ewing's, and soft tissue sarcoma (3.7%) [K. Pritchard-Jones et al. Eur. J. Cancer 42: 2183-2190 (2006)].

Although chemotherapy improves disease-free survival of patients with osteosarcomas the long-term overall survival benefit remains unproven. Chemotherapy is not efficient in chondrosarcoma and its role is currently more limited for patients with soft-tissue sarcomas. Medulloblastoma is the most common malignant brain tumour occurring in children, adolescents and young adults, with a response rate of ˜40% to temozolomide. Nevertheless, the improvement in the treatment of childhood brain tumors is particularly critical in tumor types for which outcome remains poor (such as high-grade gliomas).

There is thus an urgent and unmet need to find new antitumoral treatments in the pediatric indication.

Among the taxoid derivatives with antitumoral activity, one may cite cabazitaxel.

In particular, WO96/30355 discloses taxoids derivatives, including cabazitaxel, useful as antitumoral agents. This document also discloses a long list of other drugs that may be used as co-treatments with such taxoids.

WO2010/128258 discloses an antitumoral combination comprising cabazitaxel and capecitabine in the treatment of metastatic breast cancer for patients progressing after a previous treatment by anthracyclines and taxanes.

WO2011/051894 discloses the use of cabazitaxel in combination with prednisone or prednisolone in the treatment of prostate cancer.

The aim of the present invention is thus to provide with a new therapeutic option for treating pediatric cancers.

The aim of the present invention is to provide evidence of activity of cabazitaxel in pediatric sarcomas, using tumor models directly obtained from fresh is tumors of pediatric patients (J. J. Tentler, A. Choon Tan, C. D. Weekes, A. Jimeno, S. Leong, T. M. Pitts, J. J. Arcaroli, W. A. Messersmith and S. G. Eckhardt. Patient-derived tumour xenografts as models for oncology drug development. Nature Reviews Clinical Oncology 2012, 9: 338-350).

The present invention relates to a compound of formula (I):

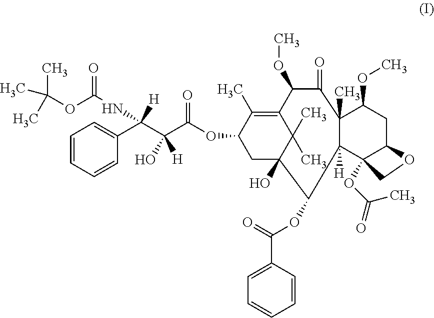 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]

which may be in the form of an anhydrous base, a hydrate or a solvate,

for its use for the treatment of pediatric cancers.

The present invention is based on an improved antitumoral activity of cabazitaxel, which may be in the form of an anhydrous base, a hydrate or a solvate, in comparison with docetaxel in preclinical pediatric models.

Indeed the present inventors have now demonstrated that the efficacy of cabazitaxel is better than that of docetaxel in this pediatric indication.

In the present invention, the term “pediatric cancers” refers to cancers or tumors occurring in children and young adults.

The present invention also relates to the above-mentioned compound for its use for the treatment of pediatric solid tumors.

In the present invention, the term “pediatric solid tumors” refers to solid tumors occurring in children and young adults.

The present invention also relates to the above-mentioned compound for its use for the treatment of high grade gliomas, such as glioblastomas.

The term “high-grade glioma” (or malignant glioma) refers to tumors that are classified as Grade III (anaplastic astrocytoma, anaplastic oligodendroglioma, anaplastic oligoastrocytoma, anaplastic ependymoma) or Grade IV (glioblastoma).

According to an embodiment, the pediatric solid tumors are chosen from the group consisting of anaplastic astrocytomas, glioblastomas, anaplastic oligodendrogliomas, oligoastrocytomas, anaplastic ependymomas, nephroblastoma, medulloblastomas, neuroblastomas, Wilm's tumors, rhabdomyosarcomas, chondrosarcomas, Ewing's sarcomas and osteosarcomas.

According to an embodiment, the present invention relates to the above-mentioned compound for its use for the treatment of rhabdomyosarcoma (such as Human Rhabdomysarcoma RH-30).

According to an embodiment, the present invention relates to the above-mentioned compound for its use for the treatment of Ewing's tumor (such as Human Ewing's sarcoma TC71, and Human Ewing's sarcoma SK-ES-1 or Human Ewing's sarcoma DM101).

According to an embodiment, the present invention relates to the above-mentioned compound for its use for the treatment of osteosarcomas (such as human osteosarcoma DM77 or human osteosarcoma DM113).

The present invention also relates to a method for treating pediatric cancers comprising the administration of a therapeutically efficient amount of the above-mentioned compound to a patient in need thereof.

Cabazitaxel is an antitumoral agent of the taxoid family and has the following formula:

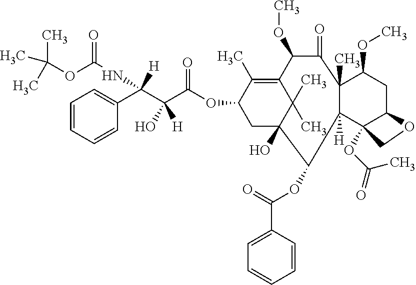 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]

It may be in the form of anhydrous base, a hydrate or a solvate.

The chemical name of cabazitaxel is 4α-acetoxy-2α-benzoyloxy-5β,20-epoxy-1β-hydroxy-7β,10β-dimethoxy-9-oxo-11-taxen-13α-yl (2R,3S)-3-tert-butoxycarbonyl-amino-2-hydroxy-3-phenylpropionate. Cabazitaxel is synonymously known as (2α,5β,7β,10β,13α)-4-acetoxy-13-({(2R,3S)-3-[(tertbutoxycarbonyl)amino]-2-hydroxy-3-phenylpropanoyl}oxy)-1-hydroxy-7,10-dimethoxy-9-oxo-5,20-epoxytax-11-en-2-yl benzoate.

This compound and a preparative method thereof are described in WO96/30355, EP0817779 and U.S. Pat. No. 5,847,170.

Cabazitaxel may be administered in base form (cf. above formula), or in the form of a hydrate. It may also be a solvate, i.e. a molecular complex characterized by the incorporation of a crystallization solvent into the crystal of the molecule of the active principle (see in this respect page 1276 of J. Pharm. Sci. 1975, 64(8), 1269-1288).

In the present invention, the above-mentioned compound may be in the form of an acetone solvate.

According to an embodiment, the acetone solvate comprises from 5% to 8% by weight of acetone.

In particular, the above-mentioned compound may be the acetone solvate described in WO2005/02846.

It may be an acetone solvate of cabazitaxel containing from 5% to 8% and preferably from 5% to 7% by weight of acetone (% means content of acetone/content of acetone+cabazitaxel×100). An average value of the acetone content is 7%, which approximately represents the acetone stoichiometry, which is 6.5% for a solvate containing one molecule of acetone.

The procedure described below allows the preparation of an acetone solvate of cabazitaxel: 940 ml of purified water are added at 20±5° C. (room temperature) to a solution of 207 g of 4α-acetoxy-2α-benzoyloxy-5β,20-epoxy-1β-hydroxy-7β,10β-dimethoxy-9-oxo-11-taxen-13α-yl (2R,3S)-3-tert-butoxycarbonylamino-2-hydroxy-3-phenylpropionate at about 92% by weight in about 2 litres of acetone, followed by seeding with a suspension of 2 g of 4α-acetoxy-2α-benzoyloxy-5β,20-epoxy-1β-hydroxy-7β,10β-dimethoxy-9-oxo-11-taxen-13α-yl(2R,3S)-3-tert-butoxycarbonyl-amino-2-hydroxy-3-phenylpro-pionate isolated from acetone/water in a mixture of 20 ml of water and 20 ml of acetone. The resulting mixture is stirred for about 10 to 22 hours, and 1.5 litres of purified water are added over 4 to 5 hours. This mixture is stirred for 60 to 90 minutes, and the suspension is then filtered under reduced pressure. The cake is washed on the filter with a solution prepared from 450 ml of is acetone and 550 ml of purified water, and then oven-dried at 55° C. under reduced pressure (0.7 kPa) for 4 hours. 197 g of 4α-acetoxy-2α-benzoyloxy-5β,20-epoxy-1β-hydroxy-7β,10β-dimethoxy-9-oxo-11-taxen-13α-yl (2R,3S)-3-tert-butoxycarbonyl-amino-2-hydroxy-3-phenylpropionate acetone containing 0.1% water and 7.2% acetone (theoretical amount: 6.5% for a stoichiometric solvate) are obtained.

In the present invention, the above-mentioned compound may be administered by parenteral route.

According to an embodiment, the compound of formula (I) is administered by intravenous route.

Cabazitaxel may be administered parenterally, such as via intravenous administration. A galenical form of cabazitaxel suitable for administration by intravenous infusion is that in which the cabazitaxel is dissolved in water in the presence of excipients chosen from surfactants, cosolvents, glucose or sodium chloride, etc. For example, a galenical form of cabazitaxel may be prepared by diluting a premix solution of cabazitaxel contained in a sterile vial (80 mg of cabazitaxel +2 ml of solvent +Polysorbate 80) with a sterile vial containing a solution of 6 ml of water and ethanol (13% by weight of 95% ethanol) in order to obtain 8 ml of a solution ready to be rediluted in a perfusion bag. The concentration of cabazitaxel in this ready-to-redilute solution is about 10 mg/ml. The perfusion is then prepared by injecting the appropriate amount of this ready-to-redilute solution into the perfusion bag containing water and glucose (about 5%) or sodium chloride (about 0.9%).

Antitumor Activity

The better antitumor activity of cabazitaxel as compared to docetaxel according to the invention is demonstrated by the head to head evaluation at same dosages and/or at equi-toxic dosages in low passage patient-derived pediatric cancer xenografts or in pediatric cancer models.

In the reported examples supporting this invention, vials of the clinical formulation of cabazitaxel and docetaxel were used. Docetaxel was diluted into 0.9% sodium chloride. Each vial of cabazitaxel, 60 mg/1.5 mL was first mixed with the entire contents of supplied diluent [13% (w/w) aqueous solution of ethanol]. The resultant solution contains 10 mg/mL of cabazitaxel. Stock solution of cabazitaxel was then diluted in 0.9% sodium chloride.

This efficacy may be quantified, for example, as changes in tumor volume for each treated (T) and control (C) group, which are calculated for each animal and each day by subtracting the tumor volume on the day of first treatment (staging day) from the tumor volume on the specified observation day. This allows calculating the tumor growth inhibition: ΔT/ΔC=(median delta T/median delta C)×100. Individual tumor volume changes from baseline are thereafter analyzed by a non-parametric two-way ANOVA-TYPE (with factors: group and repeated days) followed by a post-hoc contrasts analysis, with Bonferroni-Holm adjustment for multiplicity, comparing all treated groups to the control group. Additionally, a non parametric two-way ANOVA-TYPE (with factors: treated group and repeated days) was performed and followed by a contrast analysis, with Bonferroni-Holm adjustment for multiplicity, to compare at each day the effects of docetaxel and cabazitaxel when administered at the same dose or at equi-toxic doses. A probability less than 5% (p<0.05) was considered as significant.

Based on the National Cancer Institute (NCl) standards, a ΔT/ΔC≦40% is the minimal level required to declare activity.

The tumor doubling time (in days; Td) was estimated from the plot of the log linear growth of the control group tumors in exponential growth (100 to 1000 mm³ range) [T. H. Corbett et al., Cancer, 40: 2660-2680 (1977); F. M. Schabel et al., Cancer Drug Development, Part B, Methods in Cancer Research, 17: 3-51, New York, Academic Press Inc. (1979)].

This efficacy may also be quantified by the number of tumor regressions observed after therapy. Individual mice reporting a tumor volume ≦50% of the Day 0 measurement for two consecutive measurements over a seven day period were considered partial responders (PR). Individual mice lacking palpable tumors (<4×4 mm² for two consecutive measurements over a seven day period) were classified as complete responders (CR); a CR that persisted until study completion was considered a tumor-free survivor (TFS).

Efficacy could also be determined at study completion, using tumor growth delay (T-C) in days, which is calculated using the median time to endpoint (MTTE) value for each treatment (T) group versus control (C). A Log Rank multiple comparison test with Bonferroni-Holm adjustment for multiplicity was applied on individual TTE to compare the treated groups to the control group.

The efficacy of cabazitaxel in comparison with docetaxel on pediatric patient-derived tumor xenografts was determined experimentally in the following manner:

The animals subjected to the experiment are subcutaneously grafted unilaterally with approximately 30 mg of a tumor fragment from low passage pediatric patient-derived tumor xenografts. The animals are implanted with a human patient-derived pediatric tumor xenografted in immuno-compromised mice (Harlan; nu/nu). Several days post tumor implantation, mice are randomized according to their tumor burden to the different groups of treatments and controls. The agents are dosed intravenously at 5.8, 9.3, 15 or 24.2 mg/kg every 4 days for a total of 3 doses (q4d×3) to mice bearing a tumor burden at start of therapy (day 0) ranged from 125 to 250 mm³.

Beginning Day 0, animals were observed daily and weighed twice weekly using a digital scale; data including individual and mean gram weights (Mean We±SD), mean percent weight change versus Day 0 were recorded for each group. Animal deaths were recorded daily and designated as drug-related (D), technical (T), tumor related (B), or unknown (U) based on weight loss and gross observation; single agent or combination groups reporting a mean >20% for a period of 7 days and/or >10% mortality were considered above the maximum tolerated dose (MTD) for that treatment on the evaluated regimen.

The efficacy of cabazitaxel in comparison with docetaxel on pediatric solid tumors was determined experimentally in the following manner:

The animals subjected to the experiment are subcutaneously grafted unilaterally with approximately 30 mg of a tumor fragment on day 0. The animals are implanted with a human tumor xenografted in immunocompromized mice. Several days post tumor implantation, mice are randomized according to their body weight to the different groups of treatments and controls. The animals are observed every day. The different animal groups are weighed daily during treatment until the maximum weight loss is reached and subsequent full weight recovery has occurred. The groups are then weighed once or twice a week until the end of the trial.

The tumors are measured 1 to 5 times a week, depending on the tumor doubling time, until the tumor reaches approximately 1,000 mm³, or until the animal dies (if this occurs before the tumor reaches 1,000 mm³). The animals are necropsied immediately after euthanasia or death.

The antitumor activity is determined in accordance with the different parameters recorded.

### DESCRIPTION OF THE FIGURES

FIG. 1 represents the body weight change during the evaluation of the antitumor activity of cabazitaxel and docetaxel against human RH-30 bearing SCID female mice (example 1). Curves represent means at each day for each group.

It represents the body weight change (%) over time (days post-implantation).

The curve with continuous line corresponds to control; the curve with dotted line ( - - ) corresponds to docetaxel at 14.5 mg/kg; the curve with continuous line and a white triangle (Δ) corresponds to docetaxel at 9 mg/kg; the curve with continuous line and a white circle (◯) corresponds to docetaxel at 5.6 mg/kg; the curve with continuous line and a white square (□) corresponds to docetaxel at 3.5 mg/kg; the curve with dotted line ( - - - ) corresponds to cabazitaxel at 14.5 mg/kg; the curve with dotted line and a black triangle (▴) corresponds to cabazitaxel at 9 mg/kg; the curve with dotted line and a black circle () corresponds to cabazitaxel at 5.6 mg/kg; the curve with dotted line and a black square (▪) corresponds to cabazitaxel at 3.5 mg/kg; and the black triangles indicate the treatment IV.

FIG. 2 represents the antitumor activity of cabazitaxel and docetaxel against human RH-30 bearing SCID female mice (example 1). Curves represent medians at each day for each group.

It represents the tumor volume (mm³) over time (days post-implantation).

The curve with continuous line corresponds to control; the curve with dotted line ( - - ) corresponds to docetaxel at 14.5 mg/kg; the curve with continuous line and a white triangle (Δ) corresponds to docetaxel at 9 mg/kg; the curve with continuous line and a white circle (◯) corresponds to docetaxel at 5.6 mg/kg; the curve with continuous line and a white square (□) corresponds to docetaxel at 3.5 mg/kg; the curve with dotted line ( - - - ) corresponds to cabazitaxel at 14.5 mg/kg; the curve with dotted line and a black triangle (▴) corresponds to cabazitaxel at 9 mg/kg; the curve with dotted line and a black circle () corresponds to cabazitaxel at 5.6 mg/kg; the curve with dotted line and a black square (▪) corresponds to cabazitaxel at 3.5 mg/kg; and the black triangles indicate the treatment IV.

FIG. 3 represents the body weight change during the evaluation of the antitumor activity of cabazitaxel and docetaxel against human TC-71 bearing SCID female mice (example 2). Curves represent means at each day for each group.

It represents the body weight change (%) over time (days post-implantation).

The curve with continuous line corresponds to control; the curve with dotted line ( - - ) corresponds to docetaxel at 14.5 mg/kg; the curve with continuous line and a white triangle (Δ) corresponds to docetaxel at 9 mg/kg; the curve with continuous line and a white circle (◯) corresponds to docetaxel at 5.6 mg/kg; the curve with continuous line and a white square (□) corresponds to docetaxel at 3.5 mg/kg; the curve with dotted line ( - - - ) corresponds to cabazitaxel at 14.5 mg/kg; the curve with dotted line and a black triangle (▴) corresponds to cabazitaxel at 9 mg/kg; the curve with dotted line and a black circle () corresponds to cabazitaxel at 5.6 mg/kg; the curve with dotted line and a black square (▪) corresponds to cabazitaxel at 3.5 mg/kg; and the black triangles indicate the treatment IV.

FIG. 4 represents the antitumor activity of cabazitaxel and docetaxel against human TC-71 bearing SCID female mice (example 2). Curves represent medians at each day for each group.

It represents the tumor volume (mm³) over time (days post-implantation).

The curve with continuous line corresponds to control; the curve with dotted line ( - - ) corresponds to docetaxel at 14.5 mg/kg; the curve with continuous line and a white triangle (Δ) corresponds to docetaxel at 9 mg/kg; the curve with continuous line and a white circle (◯) corresponds to docetaxel at 5.6 mg/kg; the curve with continuous line and a white square (□) corresponds to docetaxel at 3.5 mg/kg; the curve with dotted line ( - - - ) corresponds to cabazitaxel at 14.5 mg/kg; the curve with dotted line and a black triangle (▴) corresponds to cabazitaxel at 9 mg/kg; the curve with dotted line and a black circle () corresponds to cabazitaxel at 5.6 mg/kg; the curve with dotted line and a black square (▪) corresponds to cabazitaxel at 3.5 mg/kg; and the black triangles indicate the treatment IV.

FIG. 5 represents the body weight change during the evaluation of the antitumor activity of cabazitaxel and docetaxel against human SK-ES-1 bearing SCID female mice (example 3). Curves represent means at each day for each group.

It represents the body weight change (%) over time (days post-implantation).

The curve with continuous line corresponds to control; the curve with dotted line ( - - ) corresponds to docetaxel at 14.5 mg/kg; the curve with continuous line and a white triangle (Δ) corresponds to docetaxel at 9 mg/kg; the curve with continuous line and a white circle (◯) corresponds to docetaxel at 5.6 mg/kg; the curve with continuous line and a white square (□) corresponds to docetaxel at 3.5 mg/kg; the curve with dotted line ( - - - ) corresponds to cabazitaxel at 14.5 mg/kg; the curve with dotted line and a black triangle (▴) corresponds to cabazitaxel at 9 mg/kg; the curve with dotted line and a black circle (▪) corresponds to cabazitaxel at 5.6 mg/kg; the curve with dotted line and a black square (▪) corresponds to cabazitaxel at 3.5 mg/kg; and the black triangles indicate the treatment IV.

FIG. 6 represents the antitumor activity of cabazitaxel and docetaxel against human SK-ES-1 bearing SCID female mice (example 3). Curves represent medians at each day for each group.

It represents the tumor volume (mm³) over time (days post-implantation).

The curve with continuous line corresponds to control; the curve with dotted line ( - - ) corresponds to docetaxel at 14.5 mg/kg; the curve with continuous line and a white triangle (Δ) corresponds to docetaxel at 9 mg/kg; the curve with continuous line and a white circle (◯) corresponds to docetaxel at 5.6 mg/kg; the curve with continuous line and a white square (□) corresponds to docetaxel at 3.5 mg/kg; the curve with dotted line ( - - - ) corresponds to cabazitaxel at 14.5 mg/kg; the curve with dotted line and a black triangle (▴) corresponds to cabazitaxel at 9 mg/kg; the curve with dotted line and a black circle () corresponds to cabazitaxel at 5.6 mg/kg; the curve with dotted line and a black square (▪) corresponds to cabazitaxel at 3.5 mg/kg; and the black triangles indicate the treatment IV.

FIG. 7 represents the antitumor activity of cabazitaxel and docetaxel against human DM77 osteosarcoma in nude female mice (example 4). Curves represent medians at each day for each group.

It represents the tumor volume (mm³) over time (days post first treatment).

The curve with continuous line corresponds to control; the curve with dotted line ( - - ) corresponds to docetaxel at 24.2 mg/kg; the curve with continuous line and a white triangle (Δ) corresponds to docetaxel at 15 mg/kg; the curve with continuous line and a white circle (◯) corresponds to docetaxel at 9.3 mg/kg; the curve with continuous line and a white square (□) corresponds to docetaxel at 5.8 mg/kg; the curve with dotted line ( - - - ) corresponds to cabazitaxel at 24.2 mg/kg; the curve with dotted line and a black triangle (▴) corresponds to cabazitaxel at 15 mg/kg; the curve with dotted line and a black circle () corresponds to cabazitaxel at 9.3 mg/kg; the curve with dotted line and a black square (▪) corresponds to cabazitaxel at 5.8 mg/kg; and the black triangles indicate the IV treatment.

FIG. 8 represents the antitumor activity of cabazitaxel and docetaxel against human DM113 osteosarcoma in nude female mice (example 5). Curves represent medians at each day for each group.

It represents the tumor volume (mm³) over time (days post first treatment).

The curve with continuous line corresponds to control; the curve with dotted line ( - - ) corresponds to docetaxel at 24.2 mg/kg; the curve with continuous line and a white triangle (Δ) corresponds to docetaxel at 15 mg/kg; the curve with continuous line and a white circle (◯) corresponds to docetaxel at 9.3 mg/kg; the curve with continuous line and a white square (□) corresponds to docetaxel at 5.8 mg/kg; the curve with dotted line ( - - - ) corresponds to cabazitaxel at 24.2 mg/kg; the curve with dotted line and a black triangle (▴) corresponds to cabazitaxel at 15 mg/kg; the curve with dotted line and a black circle () corresponds to cabazitaxel at 9.3 mg/kg; the curve with dotted line and a black square (▪) corresponds to cabazitaxel at 5.8 mg/kg; and the black triangles indicate the IV treatment.

FIG. 9 represents the antitumor activity of cabazitaxel and docetaxel against human DM101 Ewing's sarcoma in nude female mice (example 6). Curves represent medians at each day for each group.

It represents the tumor volume (mm³) over time (days post first treatment).

The curve with continuous line corresponds to control; the curve with dotted line ( - - ) corresponds to docetaxel at 24.2 mg/kg; the curve with continuous line and a white triangle (Δ) corresponds to docetaxel at 15 mg/kg; the curve with continuous line and a white circle (◯) corresponds to docetaxel at 9.3 mg/kg; the curve with continuous line and a white square (□) corresponds to docetaxel at 5.8 mg/kg; the curve with dotted line ( - - - ) corresponds to cabazitaxel at 24.2 mg/kg; the curve with dotted line and a black triangle (▴) corresponds to cabazitaxel at 15 mg/kg; the curve with dotted line and a black circle () corresponds to cabazitaxel at 9.3 mg/kg; the curve with dotted line and a black square (▪) corresponds to cabazitaxel at 5.8 mg/kg; and the black triangles indicate the IV treatment.

The better antitumor activity of cabazitaxel as compared to docetaxel, according to the invention, is demonstrated as illustrated in the 6 following examples.

### EXAMPLE 1

### Antitumor Activity of Cabazitaxel and Docetaxel Against Human Rhabdomyosarcoma RH-30 in SCID Female Mice

In this example, the better antitumor activity of cabazitaxel as compared to docetaxel for tumor growth inhibition was demonstrated in vivo.

The selected tumor model was a human rhabdomyosarcoma RH-30, xenografted in SCID mice [Douglass E C, et al. Cytogenet Cell Genet. 1987; 45(3-4):14855.].

Cabazitaxel and docetaxel were weighed for each treatment and dissolved in ethanol. Treatment solutions were prepared first by mixing 1 volume of ethanolic stock solution and 1 volume of polysorbate 80, then by adding 18 volumes of glucose 5% in water.

Cabazitaxel and docetaxel were administered intravenously on days 14 and 18 after tumor implantation.

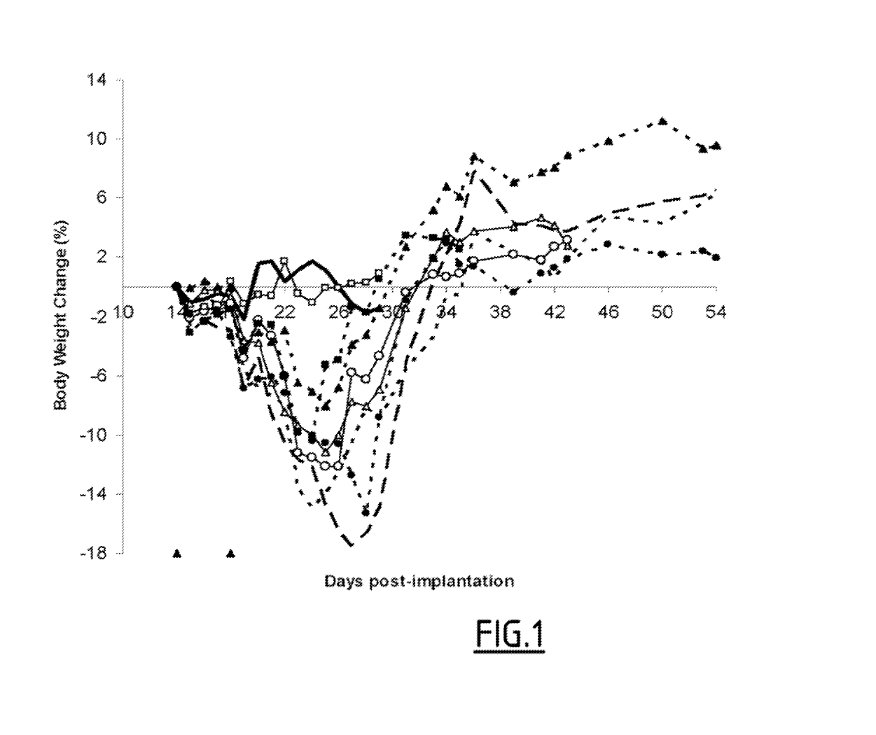

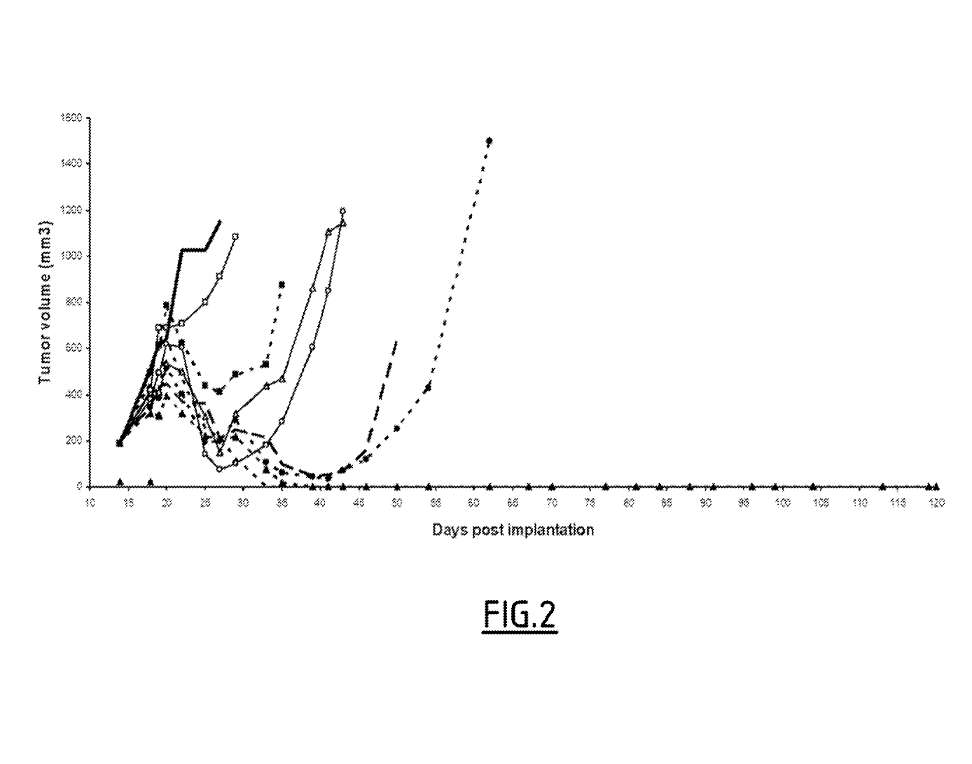

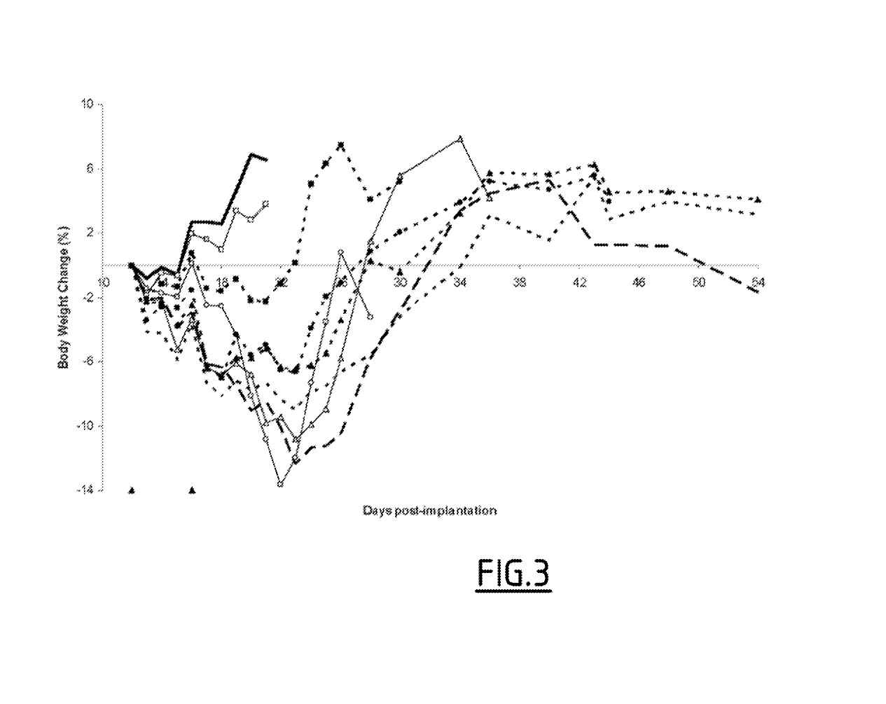

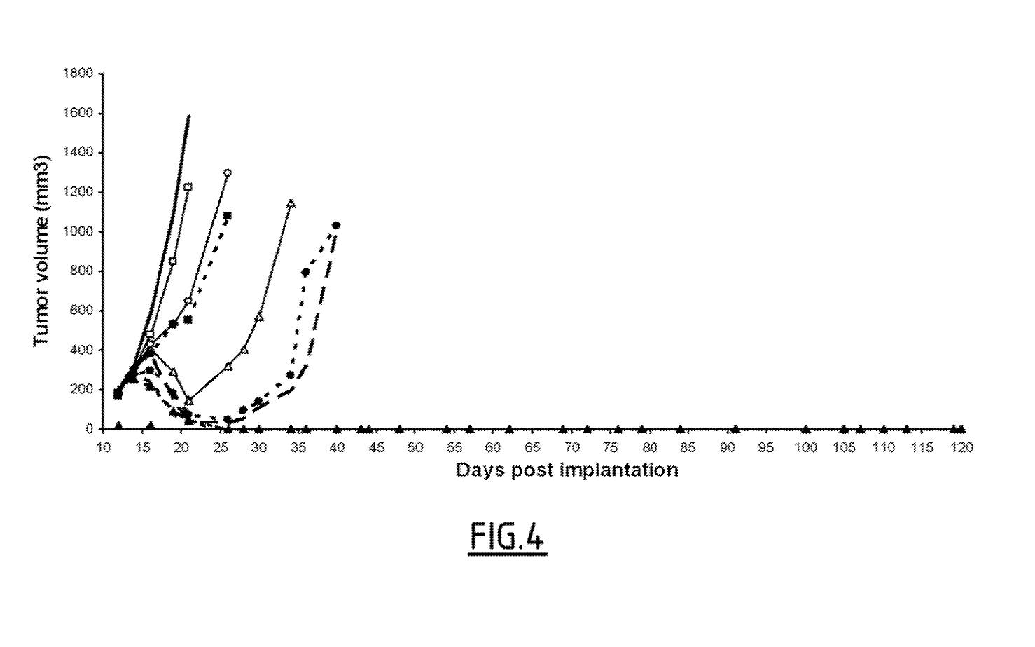

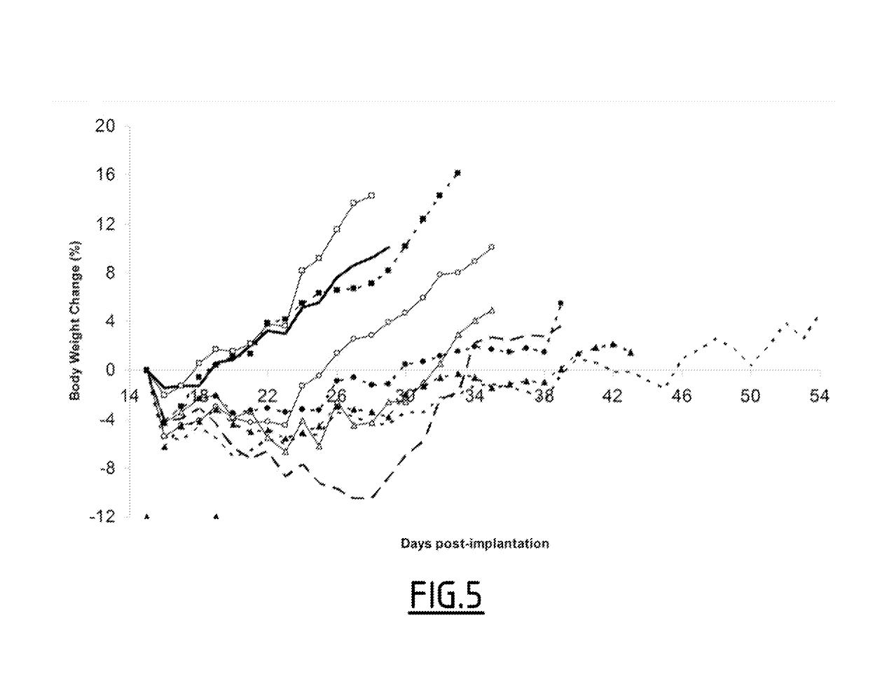

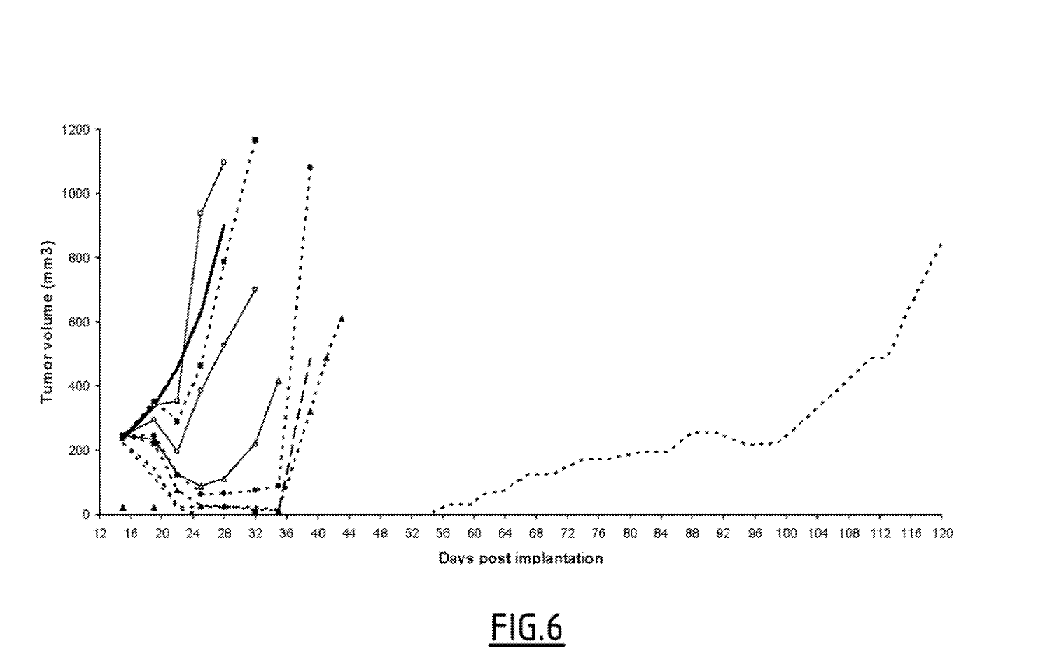

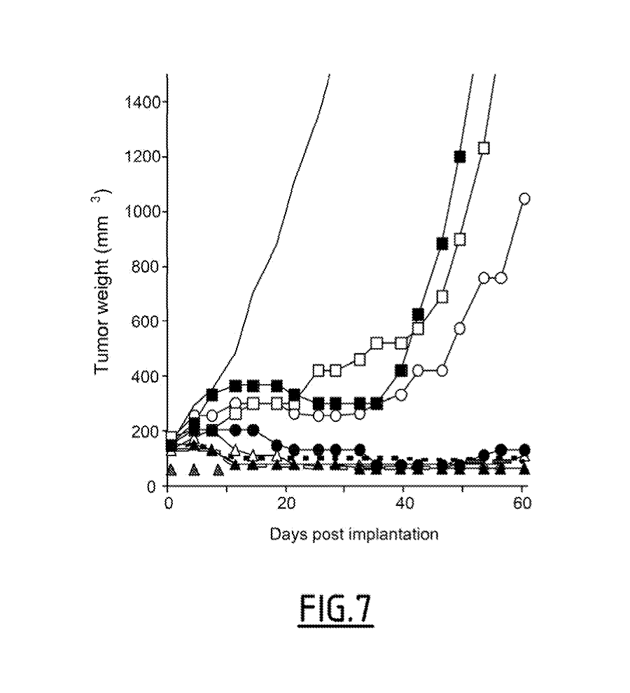

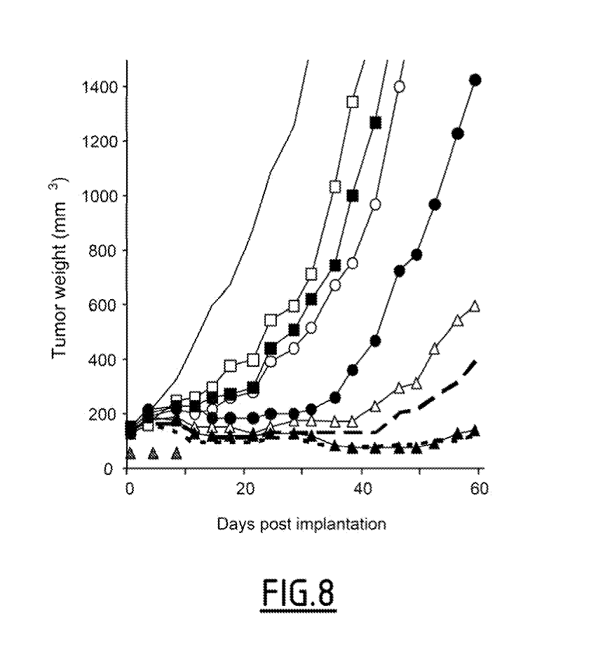

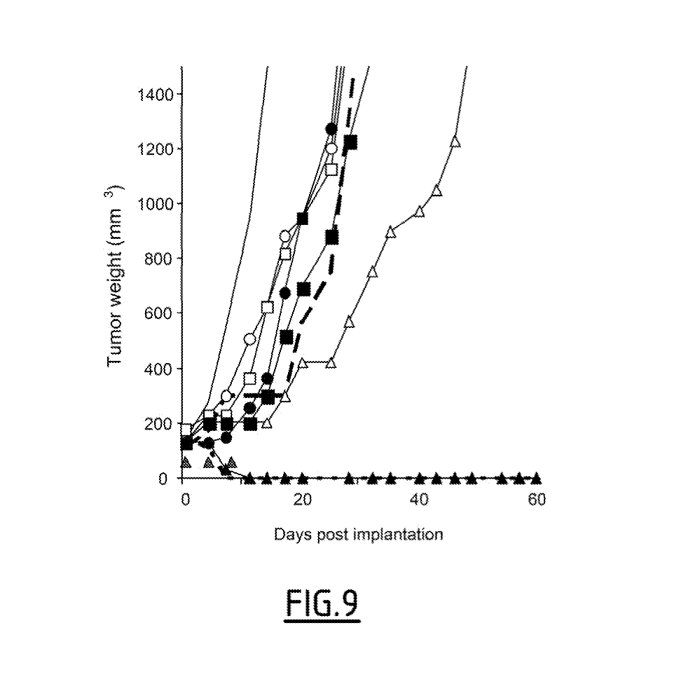

The results of the experiments are reported below in Tables 1, 2 & 3 and in FIGS. 1 & 2.

The tumor doubling time (in days; Td) was estimated from the plot of the log linear growth of the control group tumors in exponential growth (100 to 1,000 mm³ range) and the number of tumor regressions observed after therapy. Tumor doubling time was 3.2 days.

The following end points were used:
Toxicity was declared at dosages inducing >20% body weight loss or >10% drug death;
Relative tumor growth inhibition was determined on day 27 post tumor implantation when the median tumor size in the control group was 1148 mm³;
Antitumor efficacy was determined by calculating the ΔT/ΔC value in percent, according to the above mentioned formula;
Tumor regressions (as explained above);
Statistical analysis performed as explained above.

TABLE 1
Evaluation of the efficacy of docetaxel and cabazitaxel in SCID female mice bearing human rhabdomyosarcoma RH-30.

| | | | | | | Average | | | | | | | |
|---|---|---|---|---|---|---|---|---|---|---|---|---|---|
| | Route/ | Dosage | | | | body weight | | | | | Tumor | | |
| | Dosage | in mg/kg | | | Drug | change in % | | | | | free | | |
| | in mL/kg | per | | | death | per mouse | | ΔT/ΔC | | | survivors | | Biological |
| | per | injection | | Schedule | (Day of | at nadir | | in % | Regressions | | at day | P value | interpre- |
| Agent | injection | (total dose) | | in days | death) | (day of nadir) | | ( day 27) | Partial | Complete | 120 | (Day 27)^{a} | tation |
| CABAZI- | IV (16) | 14.5 | (29) | 14; 18 | 0/6 | −15.0 | (24) | <0 | 6/6 | 6/6 | 6/6 | p < 0.0001 | Highly |
| TAXEL | | | | | | | | | | | | | active |
| | | 9.0 | (18) | | 0/6 | −8.0 | (25) | 7 | 6/6 | 6/6 | 5/6 | p < 0.0001 | Very |
| | | | | | | | | | | | | | active |
| | | 5.6 | (11.2) | | 0/6 | −15.3 | (28) | 0 | 5/6 | 2/6 | 0/6 | p < 0.0001 | Highly |
| | | | | | | | | | | | | | active |
| | | 3.5 | (7.0) | | 0/6 | −10.1 | (24) | 24 | 0/6 | 0/6 | 0/6 | p < 0.0001 | Active |
| DOCE- | IV (16) | 14.5 | (29) | 14; 18 | 0/6 | −17.6 | (27) | 1 | 5/6 | 3/6 | 0/6 | p < 0.0001 | Very |
| TAXEL | | | | | | | | | | | | | active |
| | | 9.0 | (18) | | 0/6 | −11.2 | (25) | <0 | 2/6 | 0/6 | 0/6 | p < 0.0001 | Highly |
| | | | | | | | | | | | | | active |
| | | 5.6 | (11.2) | | 0/6 | −12.1 | (25) | <0 | 4/6 | 0/6 | 0/6 | p < 0.0001 | Highly |
| | | | | | | | | | | | | | active |
| | | 3.5 | (7.0) | | 0/6 | −1.8 | (15) | 77 | 0/6 | 0/6 | 0/6 | p = 0.5534 | Inactive |

| | | | | | | | | | | |
|---|---|---|---|---|---|---|---|---|---|---|
| Control | — | — | — | 0/8 | −2.2 | (19) | — | 0/8 | 0/8 | 0/8 |

Tumor doubling time = 3.2 days. Tumor size at start of therapy was 108-392 mm³, with a median tumor burden per group of 188-198 mm³.
Mice average weight: Due to body weight heterogeneity (range: DOCETAXEL = 19.73-24.51 g; CAZABITAXEL = 20.54-24.72 g) dosages were adjusted to the individual body weights.
Abbreviations used, ΔT/ΔC = ratio of median tumor volume changes from baseline between treated and control groups.
^{a)}Statistical analysis: p-value obtained with a contrast analysis versus control with Bonferroni-Holm adjustment for multiplicity after Anova-Type on tumor volume changes from baseline.

The median tumor burden at start of therapy was 188 to 198 mm³. Cabazitaxel and docetaxel were administered as single agents by IV tail vein injection on day 14 and day 18 post tumor at the following doses: 14.5, 9.0, 5.6 and 3.5 mg/kg per injection (Table 1).

Cabazitaxel and docetaxel were well tolerated, with a maximum 15.3% bwl on day 28 for cabazitaxel and 17.6% bwl on day 27 for docetaxel (Table 1 and FIG. 1).

Cabazitaxel and docetaxel were both highly active, ΔT/ΔC≦0% on day 27 (p<0.0001) at 14.5 and 5.6 mg/kg per injection for cabazitaxel and 9.0 and 5.6 mg/kg per injection for docetaxel.

Cabazitaxel at 9.0 mg/kg per injection was very active (ΔT/ΔC=7% on day 27, p<0.0001) and docetaxel at 14.5 mg/kg per injection were also very active (ΔT/ΔC=1% on day 27, p<0.0001).

At 3.5 mg/kg per injection, cabazitaxel was still active (ΔT/ΔC=24% on day 27, p<0.0001), while docetaxel was inactive (ΔT/ΔC>40% on day 27, NS) (Table 1).

The effect of cabazitaxel was significant in comparison with control on days 19, 22, 25 and 27 at 14.5 mg/kg per injection, from day 18 to day 27 at 9 mg/kg per injection, at days 18, 19, 22, 25 and 27 at 5.6 mg/kg per injection, on days 25 and 27 at 3.5 mg/kg per injection.

Global p values were p<0.0001, p<0.0001, p<0.0001 & p=0.0473 respectively for each dose (Table 2 and FIG. 2).

In this study, docetaxel had a significant effect in comparison with control on days 19, 22, 25 and 27 at 14.5 and 9 mg/kg per injection, on days 25 and 27 at 5.6 mg/kg per injection. Global p values were p<0.0001, p<0.0001 & p=0.0005, respective for each dose (Table 2 and FIG. 2).

TABLE 2
Antitumor activity of cabazitaxel and docetaxel against human
rhabdomyosarcoma RH-30 bearing SCID mice: Comparison of each agent
versus control group.
Tumor volume changes from baseline:
Median (nMad) and Anova-Type followed by a contrast analysis versus control on tumor
volume changes from baseline

| | Day | | | | | | |
|---|---|---|---|---|---|---|---|
| Group | Global | 18 | 19 | 20 | 22 | 25 | 27 |
| Control | — | 327 (83) | 437 | 403 | 852.5 | 757.5 | 956.5 |
| | | n = 8 | (149.7) | (106.7) | (418.8) | (281.7) | (588.6) |
| | | | n = 8 | n = 8 | n = 8 | n = 8 | n = 8 |
| | — | — | — | — | — | — | — |
| Cabazitaxel | — | 217.5 | 146 | 359 | 272 | 86.5 | −13.5 |
| 14.5 mg/kg | | (87.5) | (138.6) | (285.4) | (180.1) | (318) | (281) |
| | | n = 6 | n = 6 | n = 6 | n = 6 | n = 6 | n = 6 |
| | p < .0001 | p = 0.0996 | p = 0.0012 | p = 0.5335 | p = 0.0071 | p < .0001 | p < .0001 |
| Cabazitaxel 9 | — | 138.5 | 139.5 | 215.5 | 129.5 | 78.5 | 62.5 |
| mg/kg | | (34.8) | (48.2) | (88.2) | (45.2) | (174.9) | (102.3) |
| | | n = 6 | n = 6 | n = 6 | n = 6 | n = 6 | n = 6 |
| | p < .0001 | p = 0.0047 | p < .0001 | p = 0.0042 | p < .0001 | p < .0001 | p < .0001 |
| Cabazitaxel | — | 164 (30.4) | 203 (80.1) | 302.5 | 192.5 | 22 | 0 (130.5) |
| 5.6 mg/kg | | n = 6 | n = 6 | (220.2) | (196.4) | (147.5) | n = 6 |
| | | | | n = 6 | n = 6 | n = 6 | |
| | p < .0001 | p = 0.0076 | p = 0.0003 | p = 0.1708 | p = 0.0016 | p < .0001 | p < .0001 |
| Cabazitaxel | — | 307 (35.6) | 433.5 | 601 | 418 (258) | 280 | 229 |
| 3.5 mg/kg | | n = 6 | (232) | (114.2) | n = 6 | (168.3) | (78.6) |
| | | | n = 6 | n = 6 | | n = 6 | n = 6 |
| | p = 0.0473 | p = 0.8325 | p = 1.0000 | p = 1.0000 | p = 0.2529 | p = 0.0043 | p < .0001 |
| Docetaxel | — | 166.5 | 195 (83) | 247 (126) | 172 (64.5) | 154.5 | 13 (67.5) |
| 14.5 mg/kg | | (87.5) | n = 6 | n = 6 | n = 6 | (54.9) | n = 6 |
| | | n = 6 | | | | n = 6 | |
| | p < .0001 | p = 0.0828 | p = 0.0042 | p = 0.1178 | p = 0.0009 | p < .0001 | p < .0001 |
| Docetaxel | — | 202 (98.6) | 202 (71.9) | 325 | 290.5 | 115.5 | −50.5 |
| 9 mg/kg | | n = 6 | n = 6 | (181.6) | (139.4) | (120.8) | (60) |
| | | | | n = 6 | n = 6 | n = 6 | n = 6 |
| | p < .0001 | p = 0.3352 | p = 0.0293 | p = 0.6188 | p = 0.027 | p < .0001 | p < .0001 |
| Docetaxel | — | 218 (84.5) | 289 (57.1) | 409.5 | 405.5 | −30.5 | −73 |
| 5.6 mg/kg | | n = 6 | n = 6 | (109.7) | (226.8) | (68.2) | (51.1) |
| | | | | n = 6 | n = 6 | n = 6 | n = 6 |
| | p = 0.0005 | p = 0.604 | p = 0.6497 | p = 1.0000 | p = 0.2529 | p < .0001 | p < .0001 |
| Docetaxel | — | 236.5 | 477 | 475 | 495.5 | 621.5 | 736 |
| 3.5 mg/kg | | (125.3) | (157.9) | (198.7) | (276.5) | (318) | (288.4) |
| | | n = 6 | n = 6 | n = 6 | n = 6 | n = 6 | n = 6 |
| | p = 0.0473 | p = 0.8325 | p = 1.0000 | p = 1.0000 | p = 0.2529 | p = 0.0043 | p < .0001 |

p-value: obtained with a contrast analysis versus control with Bonferroni-Holm adjustment for multiplicity after Anova-Type on tumor volume changes from baseline

TABLE 3
Antitumor activity of cabazitaxel and docetaxel against human rhabdomyosarcoma RH-30 bearing SCID mice: Comparison of
the agents at the same dose
Tumor volume changes from baseline: Median (nMad) and Anova-Type followed by a contrast analysis on tumor volume changes from
baseline

| | | | | | | | | | | | Docetaxel | |
|---|---|---|---|---|---|---|---|---|---|---|---|---|
| | Cabazitaxel | Docetaxel | P | Cabazitaxel | Docetaxel | P | Cabazitaxel | Docetaxel | P | Cabazitaxel | 14.5 | P |
| Day | 3.5 mg/kg | 3.5 mg/kg | value | 5.6 mg/kg | 5.6 mg/kg | value | 9 mg/kg | 9 mg/kg | value | 14.5 mg/kg | mg/kg | value |
| 18 | 307 (35.6) | 236.5 | 0.4525 | 164 (30.4) | 218 (84.5) | 0.0294 | 138.5 (34.8) | 202 (98.6) | 0.0891 | 217.5 (87.5) | 166.5 | 0.9728 |
| | n = 6 | (125.3) | | n = 6 | n = 6 | | n = 6 | n = 6 | | n = 6 | (87.5) | |
| | | n = 6 | | | | | | | | | n = 6 | |
| 19 | 433.5 (232) | 477 | 0.5377 | 203 (80.1) | 289 (57.1) | 0.0086 | 139.5 (48.2) | 202 (71.9) | 0.0167 | 146 (138.6) | 195 (83) | 0.6549 |
| | n = 6 | (157.9) | | n = 6 | n = 6 | | n = 6 | n = 6 | | n = 6 | n = 6 | |
| | | n = 6 | | | | | | | | | | |
| 20 | 601 (114.2) | 475 | 0.5365 | 302.5 | 409.5 | 0.0993 | 215.5 (88.2) | 325 | 0.0495 | 359 (285.4) | 247 (126) | 0.8251 |
| | n = 6 | (198.7) | | (220.2) n = 6 | (109.7) | | n = 6 | (181.6) | | n = 6 | n = 6 | |
| | | n = 6 | | | n = 6 | | | n = 6 | | | | |
| 22 | 418 (258) | 495.5 | 0.5872 | 192.5 | 405.5 | 0.1388 | 129.5 (45.2) | 290.5 | 0.0672 | 272 (180.1) | 172 | 0.7155 |
| | n = 6 | (276.5) | | (196.4) n = 6 | (226.8) | | n = 6 | (139.4) | | n = 6 | (646.5) | |
| | | n = 6 | | | n = 6 | | | n = 6 | | | n = 6 | |
| 25 | 280 (168.3) | 621.5 | 0.1899 | 22 (147.5) | −30.5 | 0.9608 | 78.5 (174.9) | 115.5 | 0.5782 | 86.5 (318) | 154.5 | 0.4871 |
| | n = 6 | (318) | | n = 6 | (68.2) | | n = 6 | (120.8) | | n = 6 | (54.9) | |
| | | n = 6 | | | n = 6 | | | n = 6 | | | n = 6 | |
| 27 | 229 (78.6) | 736 | <.0001 | 0 (130.5) | −73 (51.1) | 0.4408 | 62.5 (102.3) | −50.5 (60) | 0.8153 | −13.5 (281) | 13 (67.5) | 0.7016 |
| | n = 6 | (288.4) | | n = 6 | n = 6 | | n = 6 | n = 6 | | n = 6 | n = 6 | |
| | | n = 6 | | | | | | | | | | |
| 29 | 356.5 | 905.5 | 0.0022 | 106 (147.5) | −47 (89.7) | 0.6123 | 62.5 (91.9) | 119.5 | 0.1300 | −107 (150.5) | 87 (100.8) | 0.0712 |
| | (362.5) | (41.5) | | n = 6 | n = 6 | | n = 6 | (164.6) | | n = 6 | n = 6 | |
| | n = 6 | n = 6 | | | | | | n = 6 | | | | |
| 33 | | | | −85 (117.1) | 67 (262.4) | 0.0469 | −129 (142.3) | 272 | 0.0002 | −180 (66) | 17 (111.2) | <.0001 |
| | | | | n = 6 | n = 6 | | n = 6 | (326.9) | | n = 6 | n = 6 | |
| | | | | | | | | n = 6 | | | | |
| 35 | | | | −120 (98.6) | 129 | 0.0030 | −180 (94.9) | 253.5 | <.0001 | −194 (74.1) | −80 (63.8) | 0.0034 |
| | | | | n = 6 | (281.7) | | n = 6 | (210.5) | | n = 6 | n = 6 | |
| | | | | | n = 6 | | | n = 6 | | | | |
| 39 | | | | −139 (113.4) | 454.5 | <.0001 | −189.5 | 676 | <.0001 | −194 (74.1) | −112 | 0.0275 |
| | | | | n = 6 | (348.4) | | (66.7) n = 6 | (499.6) | | n = 6 | (57.8) | |
| | | | | | n = 6 | | | n = 6 | | | n = 6 | |
| 41 | | | | −157 (113.4) | 712 | <.0001 | −189.5 | 904.5 | <.0001 | −194 (74.1) | −96 (78.6) | 0.0264 |
| | | | | n = 6 | (361.8) | | (66.7) n = 6 | (636) | | n = 6 | n = 6 | |
| | | | | | n = 6 | | | n = 6 | | | | |
| 43 | | | | −104.5 | 1039.5 | <.0001 | −189.5 | 909 | <.0001 | −194 (74.1) | −72.5 | 0.0191 |
| | | | | (114.2) n = 6 | (526.3) | | (66.7) n = 6 | (523.4) | | n = 6 | (146) | |
| | | | | | n = 6 | | | n = 6 | | | n = 6 | |
| 46 | | | | | | | | | | −194 (74.1) | 18.5 | 0.0048 |
| | | | | | | | | | | n = 6 | (270.6) | |
| | | | | | | | | | | | n = 6 | |
| 50 | | | | | | | | | | −194 (74.1) | 485 | <.0001 |
| | | | | | | | | | | n = 6 | (487.8) | |
| | | | | | | | | | | | n = 6 | |

p-value: obtained with a contrast analysis to compare the compounds at the same tested dose after 2-way Anova-Type on tumor volume changes from baseline on the two corresponding groups

Upon comparison between cabazitaxel and docetaxel treatment at the equivalent doses, a significant difference was observed with regards to improved antitumor activity for cabazitaxel.
At 14.5 mg/kg per injection a significant difference was observed between docetaxel and cabazitaxel from day 33 to day 50.
At 9.0 mg/kg per injection a significant difference was observed on days 19, and from day 33 to 43.
At 5.6 mg/kg per injection a significant difference was observed on days 18, 19 and from day 33 to 43.
At 3.5 mg/kg per injection a significant difference was observed on days 27 and 29 (Table 3; p<0.05).

Tumor regressions were seen in 3 cabazitaxel groups 14.5 mg/kg per injection (6/6 CR), 9 mg/kg per injection (6/6 CR), and 5.6 mg/kg per injection (2/6 CR, 5/6 PR), and TFS (Tumor Free Survivors) on day 120 were only obtained post treatment with cabazitaxel at 14.5 mg/kg per injection (6/6), and at 9 mg/kg per injection (5/6).

In comparison, 3/6 mice displayed CR and 5/6 PR at 14.5 mg/kg per injection of docetaxel without TFS, docetaxel achieving only PR at 9 (2/6) and 5.6 mg/kg per injection (4/6) (Table 1 and FIG. 2).

In conclusion, cabazitaxel is more active than docetaxel against the human pediatric tumor, rhabdomyosarcoma RH-30.

Cabazitaxel achieves 100% CR at 2 dose levels, leading to TFS, tumor regressions being also observed at the third dose level.

In comparison, docetaxel only induces CR at the highest dose tested.

### EXAMPLE 2

### Antitumor Activity of Cabazitaxel and Docetaxel Against Human Ewing'S Sarcoma TC-71 in SCID Female Mice

In this example, the better antitumor activity of cabazitaxel as compared to docetaxel for tumor growth inhibition was demonstrated in vivo.

The selected tumor model was a human Ewing's sarcoma TC-71, xenografted in SCID mice [Whang-Peng J, et al. Cancer Genet Cytogenet. 1986 Apr. 1; 21(3):185208].

Cabazitaxel and docetaxel were weighed for each treatment and dissolved in ethanol. Treatment solutions were prepared first by mixing 1 volume of ethanolic stock solution and 1 volume of polysorbate 80, then by adding 18 volumes of glucose 5% in water.

Cabazitaxel and docetaxel were administered intravenously on days 12 and 16 after tumor implantation.

The results of the experiments are reported in Tables 4, 5 & 6 and in FIGS. 3 & 4.

The Td in days was estimated from the plot of the log linear growth of the control group tumors in exponential growth (100 to 1,000 mm³ range) and the number of tumor regressions observed after therapy. Tumor doubling time was 2.5 days.

The following end points were used:
Toxicity was declared at dosages inducing ≧20% body weight loss or ≧10% drug death
Relative tumor growth inhibition was determined on day 21 post tumor implantation when the median tumor size in the control group was 1588.5 mm³.
Antitumor efficacy was determined by calculating the ΔT/ΔC value in percent, according to the above-mentioned formula;
Tumor regressions (as explained above);
Statistical analysis performed (as explained above).

TABLE 4
Evaluation of the efficacy of cabazitaxel and docetaxel in SCID female mice bearing human Ewing's sarcoma TC-71.

| | | | | | Average | | | | | | |
|---|---|---|---|---|---|---|---|---|---|---|---|
| | | | | | body weight | | | | | | |
| | Route/ | | | | change in | | | | Tumor | | |
| | Dosage | Dosage in | | | % per | | | | free | | |
| | in mL/kg | mg/kg per | Sched- | | mouse at | ΔT/ΔC | | | survivors | | |
| | per | injection | ule | Drug | nadir (day | in % | Regressions | | at day | P value | Biological |
| Agent | injection | (total dose) | in days | death | of nadir) | (day 21) | Partial | Complete | 120 | (Day 21)^{a} | interpretation |
| CABAZITAXEL | IV (16) | 14.5 (29) | 12; 16 | 0/7 | −9.0 (23) | <0 | 7/7 | 7/7 | 6/7 | p < 0.0001 | Highly Active |
| | | 9.0 (18) | | 0/7 | −7.0 (18) | <0 | 7/7 | 6/7 | 6/7 | p < 0.0001 | Highly Active |
| | | 5.6 (11.2) | | 0/7 | −7.0 (18) | <0 | 6/7 | 0/7 | 0/7 | p < 0.0001 | Highly Active |
| | | 3.5 (7.0) | | 0/7 | −2.3 (21) | 27 | 0/7 | 0/7 | 0/7 | p = 0.0047 | Active |
| DOCETAXEL | IV (16) | 14.5 (29) | 12; 16 | 0/7 | −12.4 (23) | <0 | 6/7 | 2/7 | 1/7 | p < 0.0001 | Highly Active |
| | | 9.0 (18) | | 0/7 | −10.8 (23) | <0 | 2/7 | 0/7 | 0/7 | p < 0.0001 | Highly Active |
| | | 5.6 (11.2) | | 0/7 | −13.7 (22) | 31 | 0/7 | 0/7 | 0/7 | p = 0.0400 | Active |
| | | 3.5 (7.0) | | 0/7 | −1.8 (13) | 77 | 0/7 | 0/7 | 0/7 | p = 0.9778 | Inactive |
| Control | — | — | — | 0/10 | −0.8 (13) | — | 0/10 | 0/10 | 0/10 | | |

Tumor doubling time = 2.5 days. Tumor size at start of therapy was 126-294 mm³, with a median tumor burden per group of 172-198 mm³.
Mice average weight: Due to body weight heterogeneity (range: docetaxel = 19.70-24.15 g; cabazitaxel = 19.25-25.07 g) dosages were adjusted to individual body weight.
Abbreviations used ΔT/ΔC = ratio of median tumor volume changes from baseline between treated and control groups.
^{a}Statistical analysis: p-value obtained with a contrast analysis versus control with Bonferroni-Holm adjustment for multiplicity after Anova-Type on tumor volume changes from baseline.

The median tumor burden at start of therapy was 172 to 198 mm³.

Cabazitaxel and docetaxel were administered as single agents by IV tail vein injection on day 12 and day 16 post tumor at the following doses, 14.5, 9, 5.6 and 3.5 mg/kg per injection (Table 4).

Cabazitaxel and docetaxel were well tolerated with a maximum 9% bwl on day 23 for cabazitaxel and 13.7% bwl on day 22 for docetaxel (Table 4 and FIG. 3).

Cabazitaxel and docetaxel were both highly active, ΔT/ΔC<0% on day 21 (p<0.0001) at 14.5, 9.0 and 5.6 mg/kg per injection for cabazitaxel and at 14.5 and 9.0 mg/kg per injection for docetaxel.

Cabazitaxel at 3.5 mg/kg per injection was considered active (ΔT/ΔC=27% on day 21, p=0.0047), while docetaxel at 5.6 mg/kg per injection was considered active (ΔT/ΔC=31% on day 21, p=0.0400), but inactive at 3.5 mg/kg per injection, ΔT/ΔC>40% on day 21, NS (Table 4).

TABLE 5
Antitumor activity of cabazitaxel and docetaxel against human Ewing's
sarcoma TC-71 bearing SCID mice: Comparison of each agent versus
control group.
Tumor volume changes from baseline:
Median (nMad) and Anova-Type followed by a contrast analysis
versus control on tumor volume changes from baseline

| | Day | | | | |
|---|---|---|---|---|---|
| Group | Global | 14 | 16 | 19 | 21 |
| Control | — | 157 | 399 | 917.5 | 1354.5 |
| | | (86) | (205.3) | (396.6) | (583.4) |
| | | n = 10 | n = 10 | n = 10 | n = 10 |
| | — | — | — | — | — |
| Cabazitaxel | — | 36 | 32 | −140 | −166 |
| 14.5 mg/kg | | (53.4) | (47.4) | (44.5) | (32.6) |
| | | n = 7 | n = 7 | n = 7 | n = 7 |
| | p < .0001 | p = 0.0029 | p < .0001 | p < .0001 | p < .0001 |
| Cabazitaxel | — | 54 | 52 | −105 | −166 |
| 9 mg/kg | | (43) | (91.9) | (26.7) | (57.8) |
| | | n = 7 | n = 7 | n = 7 | n = 7 |
| | p < .0001 | p = 0.012 | p < .0001 | p < .0001 | p < .0001 |
| Cabazitaxel | — | 88 | 150 | −18 | −81 |
| 5.6 mg/kg | | (29.7) | (80.1) | (46) | (28.2) |
| | | n = 7 | n = 7 | n = 7 | n = 7 |
| | p < .0001 | p = 0.2155 | p = 0.0004 | p < .0001 | p < .0001 |
| Cabazitaxel | — | 78 | 194 | 355 | 369 |
| 3.5 mg/kg | | (43) | (32.6) | (112.7) | (93.4) |
| | | n = 7 | n = 7 | n = 7 | n = 7 |
| | p = 0.0229 | p = 0.1702 | p = 0.0676 | p = 0.2377 | p = 0.0047 |
| Docetaxel | — | 96 | 154 | −72 | −130 |
| 14.5 mg/kg | | (86) | (140.8) | (115.6) | (112.7) |
| | | n = 7 | n = 7 | n = 7 | n = 7 |
| | p < .0001 | p = 0.2155 | p < .0001 | p < .0001 | p < .0001 |
| Docetaxel | — | 108 | 222 | 139 | −36 |
| 9 mg/kg | | (19.3) | (29.7) | (151.2) | (118.6) |
| | | n = 7 | n = 7 | n = 7 | n = 7 |
| | p < .0001 | p = 0.4719 | p = 0.0393 | p < .0001 | p < .0001 |
| Docetaxel | — | 116 | 268 | 371 | 416 |
| 5.6 mg/kg | | (4.4) | (17.8) | (150.5) | (146.8) |
| | | n = 7 | n = 7 | n = 6 | n = 6 |
| | p = 0.2527 | p = 0.6391 | p = 0.7707 | p = 0.3831 | p = 0.0400 |
| Docetaxel | — | 101 | 320 | 629 | 1044 |
| 3.5 mg/kg | | (26.7) | (90.4) | (200.2) | (243.1) |
| | | n = 7 | n = 7 | n = 7 | n = 7 |
| | p = 0.6891 | p = 0.6391 | p = 0.8453 | p = 0.864 | p = 0.9778 |

p-value: obtained with a contrast analysis versus control with Bonferroni-Holm adjustment for multiplicity after Anova-Type on tumor volume changes from baseline

The effect of cabazitaxel was significant in comparison with control from days 14 to 21 at 14.5 and 9.0 mg/kg per injection, for days 16, 19 and 21 at 5.6 mg/kg per injection, and on day 21 at 3.5 mg/kg per injection (Table 5 and FIG. 4).

In this study, docetaxel had a significant effect in comparison with control on days 16, 19 and 21 at 14.5 and 9 mg/kg per injection (global p values of p<0.0001; Table 5 and FIG. 4).

A significant effect was also seen on day 21 for docetaxel at 5.6 mg/kg per injection (p=0.04). Docetaxel at 3.5 mg/kg per injection had no significant effect on tumor volume changes as compared to the control group (Table 5 and FIG. 4).

TABLE 6
Antitumor activity of cabazitaxel and docetaxel against human Ewing's sarcoma TC-71 bearing SCID mice:
Comparison of the agents at the same dose.
Tumor volume changes from baseline:
Median (nMad) and Anova-Type followed by a contrast analysis on tumor volume changes from baseline

| | | | | | | | | | | | Docetaxel | |
|---|---|---|---|---|---|---|---|---|---|---|---|---|
| | Cabazitaxel | Docetaxel | P | Cabazitaxel | Docetaxel | P | Cabazitaxel | Docetaxel | P | Cabazitaxel | 14.5 | P |
| Day | −3.5 mg/kg | 3.5 mg/kg | value | 5.6 mg/kg | 5.6 mg/kg | value | 9 mg/kg | 9 mg/kg | value | 14.5 mg/kg | mg/kg | value |
| 14 | 78 (43) | 101 (26.7) | 0.1681 | 88 (29.7) | 116 (4.4) | 0.2209 | 54 (43) | 108 (19.3) | 0.0755 | 36 (53.4) | 96 (86) | 0.3339 |
| | n = 7 | n = 7 | | n = 7 | n = 7 | | n = 7 | n = 7 | | n = 7 | n = 7 | |
| 16 | 194 (32.6) | 320 (90.4) | <.0001 | 150 (80.1) | 268 (17.8) | 0.0096 | 52 (91.9) | 222 (29.7) | 0.0006 | 32 (47.4) | 154 | 0.0404 |
| | n = 7 | n = 7 | | n = 7 | n = 7 | | n = 7 | n = 7 | | n = 7 | (140.8) | |
| | | | | | | | | | | | n = 7 | |
| 19 | 355 (112.7) | 629 | 0.0031 | −18 (46) | 371 | <.0001 | −105 (26.7) | 139 | <.0001 | −140 (44.5) | −72 | 0.1164 |
| | n = 7 | (200.2) | | n = 7 | (150.5) | | n = 7 | (151.2) | | n = 7 | (115.6) | |
| | | n = 7 | | | n = 6 | | | n = 7 | | | n = 7 | |
| 21 | 369 (93.4) | 1044 | <.0001 | −81 (28.2) | 416 | <.0001 | −166 (57.8) | −36 | 0.0019 | −166 (32.6) | −130 | 0.2719 |
| | n = 7 | (243.1) | | n = 7 | (146.8) | | n = 7 | (118.6) | | n = 7 | (112.7) | |
| | | n = 7 | | | n = 6 | | | n = 7 | | | n = 7 | |
| 26 | | | | −162 (65.2) | 1022.5 | <.0001 | −196 (51.9) | 158 | <.0001 | −184 (43) | −130 | 0.1633 |
| | | | | n = 7 | (493) | | n = 7 | (173.5) | | n = 7 | (80.1) | |
| | | | | | n = 6 | | | n = 7 | | | n = 7 | |
| 28 | | | | | | | −196 (50.4) | 243 | <.0001 | −194 (47.4) | −144 | 0.0608 |
| | | | | | | | n = 7 | (244.6) | | n = 7 | (56.3) | |
| | | | | | | | | n = 7 | | | n = 7 | |
| 30 | | | | | | | −196 (50.4) | 406 (252) | <.0001 | −198 (53.4) | −126 | 0.0112 |
| | | | | | | | n = 7 | n = 7 | | n = 7 | (80.1) | |
| | | | | | | | | | | | n = 7 | |
| 34 | | | | | | | −196 (72.6) | 867 (209) | <.0001 | −198 (53.4) | −49 | 0.0048 |
| | | | | | | | n = 7 | n = 5 | | n = 7 | (260.9) | |
| | | | | | | | | | | | n = 7 | |
| 36 | | | | | | | | | | −180 (53.4) | 75 (330.6) | 0.0055 |
| | | | | | | | | | | n = 7 | n = 7 | |
| 40 | | | | | | | | | | −180 (53.4) | 763 | 0.0259 |
| | | | | | | | | | | n = 7 | (775.4) | |
| | | | | | | | | | | | n = 7 | |

p-value: obtained with a contrast analysis to compare the compounds at the same tested dose after 2-way Anova-Type on tumor volume changes from baseline on the two corresponding groups

Upon comparison between cabazitaxel and docetaxel at equivalent doses, a significant difference was observed with regards to improved antitumor activity for cabazitaxel.

At 14.5 mg/kg per injection, a significant difference was observed between cabazitaxel and docetaxel on day 16, and from day 30 to day 40.

At 9.0 mg/kg per injection, a significant difference was observed from day 16 to 34.

At 5.6 mg/kg per injection, a significant difference was observed from day 16 to 26.

At 3.5 mg/kg per injection, a significant difference was observed from days 16 to 21 (Table 6; p<0.05).

Tumor regressions and TFS were observed at the 2 highest doses of cabazitaxel, 14.5 mg/kg per injection (7/7 CR, 6/7 TFS) and 9 mg/kg per injection (6/7 CR, 7/7 PR, 6/7 TFS), 6/7 PR being achieved at 5.6 mg/kg per injection.

In comparison, CR and TFS were only obtained at the highest dose of docetaxel, 14.5 mg/kg per injection (2/7 CR, 6/7 PR, 1/7 TFS), 5/7 PR being observed at 9 mg/kg per injection (Table 4 and FIG. 4).

In conclusion, cabazitaxel is also more active than docetaxel against this second human pediatric tumor, Ewing's sarcoma TC-71.

Cabazitaxel achieves 6/7 TFS at 2 dose levels, 6/7 PR being also observed at the third dose level. In comparison, docetaxel only induces CR at the highest dose tested.

### EXAMPLE 3

### Antitumor Activity of Cabazitaxel and Docetaxel Against Human Ewing's Sarcoma SK-ES-1 in SCID Female Mice

In this example, the better antitumor activity of cabazitaxel as compared to docetaxel for tumor growth inhibition was demonstrated in vivo.

The selected tumor model was a human Ewing's sarcoma SK-ES-1, xenografted in SCID mice [Fogh J. New York: Plenum Press, 1975].

Cabazitaxel and docetaxel were weighed for each treatment and dissolved in ethanol. Treatment solutions were prepared first by mixing 1 volume of ethanolic stock solution and 1 volume of polysorbate 80, then by adding 18 volumes of glucose 5% in water.

Cabazitaxel and docetaxel were administered intravenously on days 15 and 19 after tumor implantation.

The results of the experiments are reported in Tables 7, 8 & 9 and in FIGS. 5 & 6.

The Td in days was estimated from the plot of the log linear growth of the control group tumors in exponential growth (100 to 1,000 mm³ range) and the number of tumor regressions observed after therapy.

Tumor doubling time was 6.1 days.

The following end points have been used:
Toxicity was declared at dosages inducing ≧20% body weight loss or ≧10% drug death;
Relative tumor growth inhibition was determined on day 22 post tumor implantation when the median tumor size in the control group was 456 mm³;
Antitumor efficacy was determined by calculating the ΔT/ΔC value in percent, according to the above-mentioned formula;
Tumor regressions (as explained above);
Statistical analysis performed (as explained above).

TABLE 7
Evaluation of the efficacy of cabazitaxel and docetaxel in SCID female mice bearing a model of human Ewing's sarcoma SK-ES-1.

| | Route/ | | | | Average | | | | | | |
|---|---|---|---|---|---|---|---|---|---|---|---|
| | Dosage | Dosage in | | | body weight | | | | Tumor | | |
| | in | mg/kg per | | | change in % | | | | free | | |
| | mL/kg | injection | Sched- | | per mouse at | ΔT/ΔC | | | survivors | | |
| | per | (total | ule | Drug | nadir (day of | in % | Regressions | | at day | P value | Biological |
| Agent | injection | dose) | in days | death | nadir) | (day 22) | Partial | Complete | 120 | (Day 22)^{a} | interpretation |
| CABAZITAXEL | IV (16) | 14.5 (29) | 15; 19 | 0/7 | −7.1 (20) | <0 | 7/7 | 6/7 | 3/7 | p < 0.0001 | Highly Active |
| | | 9.0 (18) | | 0/7 | −6.3. (16) | <0 | 7/7 | 0/7 | 0/7 | p < 0.0001 | Highly Active |
| | | 5.6 (11.2) | | 0/7 | −4.2 (16) | <0 | 7/7 | 0/7 | 0/7 | p < 0.0001 | Highly Active |
| | | 3.5 (7.0) | | 0/7 | −4.4 (16) | 22 | 0/7 | 0/7 | 0/7 | p = 0.0422 | Active |
| DOCETAXEL | IV (16) | 14.5 (29) | 15; 19 | 0/7 | −10.5 (27) | <0 | 7/7 | 3/7 | 0/7 | p < 0.0001 | Highly Active |
| | | 9.0 (18) | | 0/7 | −6.6 (23) | <0 | 6/7 | 0/7 | 0/7 | p < 0.0001 | Highly Active |
| | | 5.6 (11.2) | | 0/7 | −5.4 (16) | <0 | 1/7 | 0/7 | 0/7 | p = 0.0001 | Highly Active |
| | | 3.5 (7.0) | | 0/7 | −2.1 (16) | 72 | 0/7 | 0/7 | 0/7 | p = 0.0978 | Inactive |
| Control | — | — | — | 0/10 | −1.4 (16) | — | 0/10 | 0/10 | 0/10 | | |

Tumor doubling time = 6.1 days. Tumor size at start of therapy was 126-384 mm³, with a median tumor burden per group of 221-245 mm³.
Mice average weight: Due to body weight heterogeneity (range: DOCETAXEL = 19.09-26.69 g; CAZABITAXEL = 19.13-25.19 g) dosages were adjusted to individual body weight.
Abbreviations used ΔT/ΔC = ratio of median tumor volume changes from baseline between treated and control groups.
^{a}Statistical analysis: p-value obtained with a contrast analysis versus control with Bonferroni-Holm adjustment for multiplicity after Anova-Type on tumor volume changes from baseline.

The median tumor burden at start of therapy was 221 to 245 mm³.

Cabazitaxel and docetaxel were administered as single agents by IV tail vein injection on day 15 and day 19 post tumor at the following doses, 14.5, 9.0, 5.6 and 3.5 mg/kg per injection (Table 7).

Cabazitaxel and docetaxel were well tolerated with a maximum 7.1% bwl on day 20 for cabazitaxel and 10.5% bwl on day 27 for docetaxel (Table 7 and FIG. 5).

Cabazitaxel and docetaxel were both highly active at 14.5, 9.0 and 5.6 mg/kg per injection, ΔT/ΔC<0% on day 22 (p<0.0001 for all doses).

Cabazitaxel at 3.5 mg/kg per injection was considered active (ΔT/ΔC=22% on day 22, p=0.0422), while docetaxel at 3.5 mg/kg per injection was inactive, ΔT/ΔC>40% on day 22, NS (Table 7).

TABLE 8
Antitumor activity of cabazitaxel and docetaxel against human
Ewing's sarcoma SK-ES-1 bearing SCID mice: Comparison
of each agent versus control group.
Tumor volume changes from baseline:
Median (nMad) and Anova-Type followed by a contrast analysis
versus control on tumor volume changes from baseline

| | Day | | | | |
|---|---|---|---|---|---|
| Group | Global | 19 | 22 | 25 | 28 |
| Control | — | 32 | 188.5 | 341.5 | 648.5 |
| | | (81.5) | (149) | (123.1) | (196.4) |
| | | n = 10 | n = 10 | n = 10 | n = 10 |
| | — | — | — | — | — |
| Cabazitaxel | — | −108 | −203 | −221 | −221 |
| 14.5 mg/kg | | (91.9) | (87.5) | (81.5) | (81.5) |
| | | n = 7 | n = 7 | n = 7 | n = 7 |
| | p < .0001 | p = 0.0053 | p < .0001 | p < .0001 | p < .0001 |
| Cabazitaxel | — | 25 | −137 | −227 | −227 |
| 9 mg/kg | | (37.1) | (60.8) | (100.8) | (100.8) |
| | | n = 7 | n = 7 | n = 7 | n = 7 |
| | p < .0001 | p = 1.0000 | p < .0001 | p < .0001 | p < .0001 |
| Cabazitaxel | — | −31 | −126 | −157 | −157 |
| 5.6 mg/kg | | (87.5) | (86) | (81.5) | (81.5) |
| | | n = 7 | n = 7 | n = 7 | n = 7 |
| | p < .0001 | p = 0.7871 | p < .0001 | p < .0001 | p < .0001 |
| Cabazitaxel | — | 32 | 41 | 180 | 499 |
| 3.5 mg/kg | | (207.6) | (108.2) | (100.8) | (324.7) |
| | | n = 7 | n = 7 | n = 7 | n = 7 |
| | p = 0.6074 | p = 1.0000 | p = 0.0422 | p = 0.5810 | p = 0.9384 |
| Docetaxel | — | −18 | −156 | −173 | −164 |
| 14.5 mg/kg | | (77.1) | (56.3) | (69.7) | (56.3) |
| | | n = 7 | n = 7 | n = 7 | n = 7 |
| | p < .0001 | p = 0.5639 | p < .0001 | p < .0001 | p < .0001 |
| Docetaxel | — | 0 | −101 | −126 | −126 |
| 9 mg/kg | | (37.1) | (62.3) | (32.6) | (46) |
| | | n = 7 | n = 7 | n = 7 | n = 7 |
| | p < .0001 | p = 1.0000 | p < .0001 | p < .0001 | p < .0001 |
| Docetaxel | — | 0 | −36 | 168 | 342 |
| 5.6 mg/kg | | (106.7) | (60.8) | (80.1) | (89) |
| | | n = 7 | n = 7 | n = 7 | n = 7 |
| | p = 0.0194 | p = 1.0000 | p = 0.0001 | p = 0.0359 | p = 0.1047 |
| Docetaxel | — | 52 | 136 | 712 | 900 |
| 3.5 mg/kg | | (89) | (266.9) | (29.7) | (373.6) |
| | | n = 7 | n = 7 | n = 7 | n = 7 |
| | p = 0.7742 | p = 1.0000 | p = 0.0978 | p = 0.5810 | p = 0.9384 |

p-value: obtained with a contrast analysis versus control with Bonferroni-Holm adjustment for multiplicity after Anova-Type on tumor volume changes from baseline

The effect of cabazitaxel was significant in comparison with control from days 19 to 28 at 14.5 mg/kg per injection, on days 22 to 28 at 9.0 and 5.6 mg/kg per injection. Global p values were p<0.0001 for each dose.

A significant effect was also seen on day 22 only for cabazitaxel at 3.5 mg/kg per injection (p=0.0422) (Table 8 and FIG. 6).

In this study, docetaxel had a significant effect in comparison with control on days 22 to 28 at 14.5 and 9 mg/kg per injection and on day 22 and 25 at 5.6 mg/kg per injection. Global p values were p<0.0001, p<0.001 & p=0.0194 respective for each dose (Table 8 and FIG. 6).

Docetaxel at 3.5 mg/kg per injection had no significant effect on tumor volume changes as compared to the control group.

TABLE 9
Antitumor activity of cabazitaxel and docetaxel against human Ewing's sarcoma SK-ES-1 bearing SCID mice: Comparison
of the agents at the same dose.
Tumor volume changes from baseline:
Median (nMad) and Anova-Type followed by a contrast analysis on tumor volume changes from baseline

| | | | | | | | | | | | Docetaxel | |
|---|---|---|---|---|---|---|---|---|---|---|---|---|
| | Cabazitaxel | Docetaxel | P | Cabazitaxel | Docetaxel | P | Cabazitaxel | Docetaxel | P | Cabazitaxel | 14.5 | P |
| Day | 3.5 mg/kg | 3.5 mg/kg | value | 5.6 mg/kg | 5.6 mg/kg | value | 9 mg/kg | 9 mg/kg | value | 14.5 mg/kg | mg/kg | value |
| 19 | 32 (207.6) | 52 (89) | 0.7323 | −31 (87.5) | 0 (106.7) | 0.3683 | 25 (37.1) | 0 (37.1) | 0.5214 | −108 (91.9) | −18 (77.1) | 0.4035 |
| | n = 7 | n = 7 | | n = 7 | n = 7 | | n = 7 | n = 7 | | n = 7 | n = 7 | |
| 22 | 41 (108.2) | 136 | 0.3594 | −126 (86) | −36 (60.8) | 0.0466 | −137 (60.8) | −101 | 0.1647 | −203 (87.5) | −156 | 0.2939 |
| | n = 7 | (266.9) | | n = 7 | n = 7 | | n = 7 | (62.3) | | n = 7 | (56.3) | |
| | | n = 7 | | | | | | | n = 7 | | n = 7 | |
| 25 | 180 (100.8) | 712 (29.7) | 0.0057 | −157 (81.5) | 168 (80.1) | <.0001 | −227 (100.8) | −126 | 0.0031 | −221 (81.5) | −173 | 0.6869 |
| | n = 7 | n = 7 | | n = 7 | n = 7 | | n = 7 | (32.6) | | n = 7 | (69.7) | |
| | | | | | | | | n = 7 | | | n = 7 | |
| 28 | 499 (324.7) | 900 | 0.1065 | −157 (81.5) | 342 (89) | <.0001 | −227 (100.8) | −126 (46) | 0.0005 | −221 (81.5) | −164 | 0.6382 |
| | n = 7 | (373.6) | | n = 7 | n = 7 | | n = 7 | n = 7 | | n = 7 | (56.3) | |
| | | n = 7 | | | | | | | | | n = 7 | |
| 32 | | | | −128 (112.7) | 480 | <.0001 | −231 (78.6) | −49 | <.0001 | −221 (87.5) | −162 | 0.25 |
| | | | | n = 7 | (204.6) | | n = 7 | (100.8) | | n = 7 | (60.8) | |
| | | | | | n = 7 | | | n = 7 | | | n = 7 | |
| 35 | | | | −157 (112.7) | 2005 | <.0001 | −231 (78.6) | 201 | <.0001 | −221 (87.5) | −162 | 0.257 |
| | | | | n = 7 | (1055.6) | | n = 7 | (256.5) | | n = 7 | (60.8) | |
| | | | | | n = 7 | | | n = 7 | | | n = 7 | |
| 39 | | | | | | | | | | −221 (108.2) | 290 | 0.0002 |
| | | | | | | | | | | n = 7 | (468.5) | |
| | | | | | | | | | | | n = 7 | |
| 41 | | | | | | | | | | −221 (140.8) | 274 | 0.0011 |
| | | | | | | | | | | n = 7 | (299.5) | |
| | | | | | | | | | | | n = 6 | |
| 43 | | | | | | | | | | −221 (140.8) | 427.5 | 0.0002 |
| | | | | | | | | | | n = 7 | (428.5) | |
| | | | | | | | | | | | n = 6 | |
| 45 | | | | | | | | | | −144 (222.4) | 574 | 0.0014 |
| | | | | | | | | | | n = 7 | (566.4) | |
| | | | | | | | | | | | n = 5 | |

p-value: obtained with a contrast analysis to compare the compounds at the same tested dose after 2-way Anova-Type on tumor volume changes from baseline on the two corresponding groups

Upon comparison between cabazitaxel and docetaxel at equivalent doses, a significant difference was observed with regards to improved antitumor activity for cabazitaxel.

At 14.5 mg/kg per injection, a significant difference was observed between docetaxel and cabazitaxel from day 39 to day 45.

At 9.0 mg/kg per injection, a significant difference was observed from day 25 to 35.

At 5.6 mg/kg per injection, a significant difference was observed from day 22 to 35.

At 3.5 mg/kg per injection, a significant difference was observed on day 25 only (Table 9; p<0.05).

CR and TFS were observed at the highest dose of cabazitaxel, 14.5 mg/kg per injection (6/7 CR, 7/7 PR, 3/7 TFS), 100% PR being achieved at 9 and 5.6 mg/kg per injection.

In comparison only 3/7 mice displayed CR at 14.5 mg/kg per injection of docetaxel, with 7/7 PR and no TFS on day 120. At 9 and 5.6 mg/kg per injection, docetaxel induced 6/7 and 1/7 PR, respectively (Table 7 and FIG. 6).

In conclusion, cabazitaxel is more also active than docetaxel against this third human pediatric tumor, Ewing's sarcoma SK-ES-1.

Cabazitaxel achieves 100% PR at a 3 dose levels, with 6/7 CR leading to 3/7 TFS at the highest doses tested. In comparison, docetaxel induced 3/7 CR at the highest dose tested and no TFS.

### EXAMPLE 4

### Antitumor Activity of Cabazitaxel and Docetaxel Against Human Osteosarcoma DM77 in Nude Female Mice

In this example, the better antitumor activity of cabazitaxel as compared to docetaxel for tumor growth inhibition was demonstrated in vivo.

The selected tumor model, DM77, was a low passage patient-derived tumor xenograft derived from an osteosarcoma taken from the lung of a 19 year old male patient.

The results of the experiments are reported below in Tables 10, 11 & 12 and in FIG. 7.

The tumor doubling time (in days; Td) was 6.6 days.

The following end points were used:
Toxicity was declared at dosages inducing ≧20% body weight loss or ≧10% drug death;
Antitumor efficacy was determined by calculating the ΔT/ΔC value in percent on day 21 post treatment initiation, according to the above mentioned formula;
Individual tumor volume changes from baseline were analyzed by a non-parametric two-way ANOVA-TYPE (with factors: group and repeated day from 3 to 21) followed by a post-hoc contrasts analysis, with Bonferroni-Holm adjustment for multiplicity, comparing all treated groups to the control group on day 21. Additionally, a non parametric two-way ANOVA-TYPE (with factors: treated group and repeated day from 3 to 56) was performed and followed by a contrast analysis, with Bonferroni-Holm adjustment for multiplicity, to compare at each day the effects of docetaxel and cabazitaxel when administered at the same dose or at equi-toxic doses.
At study completion, tumor growth delay (T-C) in days is calculated using the median time to endpoint (MTTE) value for each treatment (T) group versus control (C). The volume endpoint for T-C calculations was chosen to be 1400 mm³. A Log Rank multiple comparison test with Bonferroni-Holm adjustment for multiplicity was applied on individual TTE to compare the treated groups to the control group.
Tumor regressions (as explained above).

Results:

Cabazitaxel and docetaxel demonstrate anti-tumor effects compared to the control (FIG. 7 and Table 11). At day 21, a ΔT/ΔC of 14.1% or 18.5% was reported for animals treated with 5.8 mg/kg of cabazitaxel or docetaxel, respectively and 0% or 9.6% ΔT/ΔC was reported for animals treated with 9.3 mg/kg of cabazitaxel or docetaxel, respectively. Animals dosed with 15 or 24.2 mg/kg had a ΔT/ΔC lower than 0% for both test agents.

Comparison of tumor volume changes demonstrated that cabazitaxel at 9.3 mg/kg was more efficacious than docetaxel from day 25 to day 56 (Table 12). Similar results are observed when comparing the numbers of PR between treatment groups at 9.3 mg/kg (2/9 versus 0/9 PR, respectively) (Table 11).

Using weight loss as a gross indicator of toxicity, docetaxel appears to more toxic than cabazitaxel (Table 10). Docetaxel at 24.2 mg/kg was inducing an excessive body weight loss of 17% on day 14. At 15 mg/kg, docetaxel is inducing 14% body weight loss on day 11, which is comparable to the 15% body weight loss observed for cabazitaxel at 24.2 mg/kg on day 14. Alternative analysis, adjusting for the higher level of toxicity was performed (Table 12). The tumor volume changes from baseline for docetaxel at 5.8, 9.3, or 15 mg/kg were compared along time to cabazitaxel at 9.3, 15, or 24.2 mg/kg, respectively. Docetaxel was significantly different from cabazitaxel: 5.8 mg/kg docetaxel to 9.3 mg/kg cabazitaxel (from day 18) and 9.3 mg/kg docetaxel to 15 mg/kg cabazitaxel (from day 11). The comparison of tumor volume changes did not show any significant differences at the highest dosages, the study being terminated before the regrowth of the tumors.

TABLE 10
Cabazitaxel and docetaxel toxicity in nude mice bearing DM77
osteosarcoma

| | | | Weight | | | |
|---|---|---|---|---|---|---|
| | | | Change | | | |
| | Dose | | at Nadir | | Drug Deaths | |
| Treatment | (mg/kg) | Route/Schedule | % | Day | Total | Day (#) |
| Control | — | i.v./q4dx3 | — | — | — | — |
| Cabazitaxel | 5.8 | i.v./q4dx3 | −5% | 11 | 0 | — |
| | 9.3 | i.v./q4dx3 | −8% | 11 | 0 | — |
| | 15 | i.v./q4dx3 | −9% | 11 | 0 | — |
| | 24.2 | i.v./q4dx3 | −15% | 14 | 0 | — |
| Docetaxel | 5.8 | i.v./q4dx3 | −6% | 11 | 0 | — |
| | 9.3 | i.v./q4dx3 | −7% | 14 | 0 | — |
| | 15 | i.v./q4dx3 | −14% | 11 | 0 | — |
| | 24.2 | i.v./q4dx3 | −17% | 14 | 0 | — |

TABLE 11
Cabazitaxel and docetaxel antitumor activity in nude mice bearing DM77 osteosarcoma

| | | | Tumor Volume Data (Day 21) | | | | | | | |
|---|---|---|---|---|---|---|---|---|---|---|
| | Dose | | Median | | | MTTE | | T-C | | |
| Treatment | (mg/kg) | Route/Schedule | (mm³) | ΔT/ΔC % | pvalue* | (days) | pvalue** | (days) | n | #PR/CR/TFS |
| Control | — | i.v./q4dx3 | 1102.5 | | | 25 | — | — | 10 | — |
| Cabazitaxel | 5.8 | i.v./q4dx3 | 333 | 14.1 | p = 0.0006 | 49 | p = 0.0132 | 24 | 9 | 0/0/0 |
| | 9.3 | i.v./q4dx3 | 131 | 0 | p < 0.0001 | >60 | p < .0001 | >35 | 9 | 2/0/0 |
| | 15 | i.v./q4dx3 | 78 | −9.3 | p < 0.0001 | >60 | p < .0001 | >35 | 9 | 6/0/0 |
| | 24.2 | i.v./q4dx3 | 101.5 | −6.9 | p < 0.0001 | >60 | p < .0001 | >35 | 10 | 5/1/1 |
| Docetaxel | 5.8 | i.v./q4dx3 | 300 | 18.5 | p = 0.0056 | 53 | p = 0.0023 | 28 | 9 | 0/0/0 |
| | 9.3 | i.v./q4dx3 | 266 | 9.6 | p < 0.0001 | >60 | p = 0.0014 | >35 | 9 | 0/0/0 |
| | 15 | i.v./q4dx3 | 78 | −5.9 | p < 0.0001 | >60 | p < .0001 | >35 | 9 | 3/0/0 |
| | 24.2 | i.v./q4dx3 | 71.5 | −6.1 | p < 0.0001 | >60 | p < .0001 | >35 | 10 | 6/1/1 |

*Contrasts analysis versus control with Bonferroni-Holm adjustment for multiplicity following a non parametric two-way Anova-Type on tumor volume changes from baseline
**Log-Rank multiple comparisons test versus control on individuals time to event

TABLE 12
Comparison of the tumor volumes of the groups treated with cabazitaxel and docetaxel at the same dose and
at equi-toxic doses in nude mice bearing DM77 osteosarcoma

| | Median +/− nMAD (number of subject) and pvalue* | | | | | | | |
|---|---|---|---|---|---|---|---|---|
| | Cabazitaxel | Cabazitaxel | Cabazitaxel | Cabazitaxel | Docetaxel | Docetaxel | Docetaxel | Docetaxel |
| | 5.8 mg/kg | 9.3 mg/kg | 15 mg/kg | 24.2 mg/kg | 5.8 mg/kg | 9.3 mg/kg | 15 mg/kg | 24.2 mg/kg |
| DAY 4 | 47 +/− 47 | 54 +/− 54 | 0 +/− 0 (n = 9) | 22 +/− 22 | 47 +/− 47 | 87 +/− 40 | 66 +/− 66 | 0 +/− 0 |
| | (n = 9) | (n = 9) | | (n = 10) | (n = 9) | (n = 9) | (n = 9) | (n = 10) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 1.0000 | p = 1.0000 | p = 1.0000 | p = 1.0000 | p = 1.0000 | p = 1.0000 | |
| DAY 7 | 121 +/− 80 | 73 +/− 73 | 0 +/− 25 | 0 +/− 9.5 | 73 +/− 23 | 87 +/− 42 | 73 +/− 73 | 0 +/− 0 |
| | (n = 9) | (n = 9) | (n = 9) | (n = 10) | (n = 9) | (n = 9) | (n = 9) | (n = 10) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 1.0000 | p = 1.0000 | p = 1.0000 | p = 1.0000 | p = 0.5271 | p = 1.0000 | |
| DAY 11 | 162 +/− 89 | 19 +/− 35 | −53 +/− 19 | −26.5 +/− | 122 +/− 56 | 96 +/− 77 | −41 +/− 41 | −50 +/− 24 |
| | (n = 9) | (n = 9) | (n = 9) | 26.5 (n = 10) | (n = 9) | (n = 9) | (n = 9) | (n = 10) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 1.0000 | p = 1.0000 | p = 1.0000 | p = 1.0000 | p = 0.0008 | p = 1.0000 | |
| DAY 14 | 162 +/− 97 | 19 +/− 35 | −53 +/− 27 | −29.5 +/− | 129 +/− 63 | 96 +/− 69 | −41 +/− 41 | −55.5 +/− 20 |
| | (n = 9) | (n = 9) | (n = 9) | 29.5 (n = 10) | (n = 9) | (n = 9) | (n = 9) | (n = 10) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 1.0000 | p = 1.0000 | p = 1.0000 | p = 1.0000 | p < 0.0001 | p = 1.0000 | |
| DAY 18 | 195 +/− 122 | 0 +/− 33 | −73 +/− 20 | −61.5 +/− 27 | 169 +/− 116 | 96 +/− 69 | −53 +/− 17 | −55.5 +/− 20 |
| | (n = 9) | (n = 9) | (n = 9) | (n = 10) | (n = 9) | (n = 9) | (n = 9) | (n = 10) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 0.1171 | p = 1.0000 | p = 1.0000 | p = 0.0302 | p < 0.0001 | p = 1.0000 | |
| DAY 21 | 129 +/− 129 | 0 +/− 34 | −85 +/− 13 | −63 +/− 25.5 | 169 +/− 103 | 88 +/− 74 | −54 +/− 12 | −55.5 +/− 10.5 |
| | (n = 9) | (n = 9) | (n = 9) | (n = 10) | (n = 9) | (n = 9) | (n = 9) | (n = 10) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 0.1509 | p = 1.0000 | p = 1.0000 | p = 0.0175 | p < 0.0001 | p = 1.0000 | |
| DAY 25 | 96 +/− 115 | −19 +/− 26 | −85 +/− 13 | −69.5 +/− | 217 +/− 151 | 124 +/− 93 | −66 +/− 17 | −55.5 +/− 10.5 |
| | (n = 9) | (n = 9) | (n = 9) | 24.5 (n = 10) | (n = 9) | (n = 9) | (n = 9) | (n = 10) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 0.0028 | p = 1.0000 | p = 1.0000 | p = 0.0002 | p < 0.0001 | p = 1.0000 | |
| DAY 28 | 96 +/− 115 | −45 +/− 26 | −85 +/− 13 | −60 +/− 28.5 | 290 +/− 198 | 124 +/− 171 | −66 +/− 17 | −53.5 +/− 13 |
| | (n = 9) | (n = 9) | (n = 9) | (n = 10) | (n = 9) | (n = 9) | (n = 9) | (n = 10) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 0.0002 | p = 1.0000 | p = 1.0000 | p < 0.0001 | p < 0.0001 | p = 1.0000 | |
| DAY 32 | 96 +/− 109 | −45 +/− 26 | −85 +/− 19 | −60 +/− 28.5 | 332 +/− 266 | 154 +/− 201 | −66 +/− 17 | −53.5 +/− 33 |
| | (n = 9) | (n = 9) | (n = 9) | (n = 10) | (n = 9) | (n = 9) | (n = 9) | (n = 10) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p < 0.0001 | p = 1.0000 | p = 1.0000 | p < 0.0001 | p < 0.0001 | p = 1.0000 | |
| DAY 35 | 169 +/− 182 | −66 +/− 7 | −85 +/− 19 | −60 +/− 35 | 342 +/− 250 | 169 +/− 235 | −66 +/− 13 | −57.5 +/− 45.5 |
| | (n = 9) | (n = 9) | (n = 9) | (n = 10) | (n = 9) | (n = 9) | (n = 9) | (n = 10) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p < 0.0001 | p = 1.0000 | p = 1.0000 | p < 0.0001 | p < 0.0001 | p = 1.0000 | |
| DAY 39 | 239 +/− 172.5 | −66 +/− 7 | −85 +/− 38 | −60 +/− 35 | 342 +/− 121 | 202 +/− 268 | −66 +/− 13 | −57.5 +/− |
| | (n = 8) | (n = 9) | (n = 9) | (n = 10) | (n = 9) | (n = 9) | (n = 9) | 45.5 (n = 10) |
| Comparison | Docetaxel 5.8 mg/kg | Docetaxel | Docetaxel | Docetaxel | Cabazitaxel | Cabazitaxel | Cabazitaxel | |
| versus | | 9.3 mg/kg | 15 mg/kg | 24.2 mg/kg | 9.3 mg/kg | 15 mg/kg | 24.2 mg/kg | |
| | p = 1.0000 | p < 0.0001 | p = 1.0000 | p = 1.0000 | p < 0.0001 | p < 0.0001 | p = 1.0000 | |
| DAY 42 | 240 +/− 162 | −66 +/− 7 | −85 +/− 38 | −71 +/− 46.5 | 401 +/− 169.5 | 309 +/− 375 | −66 +/− 13 | −57.5 +/− |
| | (n = 6) | (n = 9) | (n = 9) | (n = 10) | (n = 8) | (n = 9) | (n = 9) | 45.5 (n = 10) |
| Comparison | Docetaxel 5.8 mg/kg | Docetaxel | Docetaxel | Docetaxel | Cabazitaxel | Cabazitaxel | Cabazitaxel | |
| versus | | 9.3 mg/kg | 15 mg/kg | 24.2 mg/kg | 9.3 mg/kg | 15 mg/kg | 24.2 mg/kg | |
| | p = 1.0000 | p < 0.0001 | p = 1.0000 | p = 0.9613 | p < 0.0001 | p < 0.0001 | p = 1.0000 | |
| DAY 46 | 364.5 +/− 226 | −66 +/− 7 | −85 +/− 38 | −71 +/− 46.5 | 546 +/− 185.5 | 309 +/− 375 | −58 +/− 11 | −57.5 +/− |
| | (n = 6) | (n = 9) | (n = 9) | (n = 10) | (n = 8) | (n = 9) | (n = 9) | 45.5 (n = 10) |
| Comparison | Docetaxel 5.8 mg/kg | Docetaxel | Docetaxel | Docetaxel | Cabazitaxel | Cabazitaxel | Cabazitaxel | |
| versus | | 9.3 mg/kg | 15 mg/kg | 24.2 mg/kg | 9.3 mg/kg | 15 mg/kg | 24.2 mg/kg | |
| | p = 1.0000 | p < 0.0001 | p = 1.0000 | p = 0.7558 | p < 0.0001 | p < 0.0001 | p = 1.0000 | |
| DAY 49 | 402 +/− 254 | −66 +/− 7 | −85 +/− 38 | −71 +/− 43 | 512 +/− 142 | 424.5 +/− | −58 +/− 11 | −48 +/− 45.5 |
| | (n = 6) | (n = 9) | (n = 9) | (n = 10) | (n = 7) | 394.5 (n = 8) | (n = 9) | (n = 10) |
| Comparison | Docetaxel 5.8 mg/kg | Docetaxel | Docetaxel | Docetaxel | Cabazitaxel | Cabazitaxel | Cabazitaxel | |
| versus | | 9.3 mg/kg | 15 mg/kg | 24.2 mg/kg | 9.3 mg/kg | 15 mg/kg | 24.2 mg/kg | |
| | p = 0.9989 | p < 0.0001 | p = 0.9989 | p = 0.4781 | p < 0.0001 | p < 0.0001 | p = 0.9989 | |
| DAY 53 | 706 +/− 516 | −47 +/− 25 | −85 +/− 38 | −64.5 +/− 45.5 | 657.5 +/− | 542 +/− 447.5 | −58 +/− 11 | −48 +/− 69.5 |
| | (n = 6) | (n = 9) | (n = 9) | (n = 10) | 211.5 (n = 6) | (n = 8) | (n = 9) | (n = 10) |
| Comparison | Docetaxel 5.8 mg/kg | Docetaxel | Docetaxel | Docetaxel | Cabazitaxel | Cabazitaxel | Cabazitaxel | |
| versus | | 9.3 mg/kg | 15 mg/kg | 24.2 mg/kg | 9.3 mg/kg | 15 mg/kg | 24.2 mg/kg | |
| | p = 0.7526 | p < 0.0001 | p = 0.7526 | p = 0.4742 | p < 0.0001 | p < 0.0001 | p = 0.7526 | |
| DAY 56 | 878 +/− 602.5 | −47 +/− 26 | −85 +/− 38 | −64.5 +/− 46.5 | 875.5 +/− | 493 +/− 238 | −58 +/− 11 | −48 +/− 69.5 |
| | (n = 6) | (n = 9) | (n = 9) | (n = 10) | 358.5 (n = 6) | (n = 7) | (n = 9) | (n = 10) |
| Comparison | Docetaxel 5.8 mg/kg | Docetaxel | Docetaxel | Docetaxel | Cabazitaxel | Cabazitaxel | Cabazitaxel | |
| versus | | 9.3 mg/kg | 15 mg/kg | 24.2 mg/kg | 9.3 mg/kg | 15 mg/kg | 24.2 mg/kg | |
| | p = 0.7397 | p < 0.0001 | p = 0.7397 | p = 0.6100 | p < 0.0001 | p < 0.0001 | p = 0.7397 | |

*Contrasts analysis with Bonferroni-Holm adjustment for multiplicity following a two-way ANOVA-TYPE on tumor volume changes from baseline to compare, at each day, the groups treated with Cabazitaxel or Docetaxel at the same dose or at equitoxic doses.

Conclusion:

Cabazitaxel and docetaxel demonstrated robust dose-dependent anti-tumor activity. Overall, dosing with 15 mg/kg and 9.3 mg/kg of cabazitaxel induces higher antitumor activity than docetaxel at an equivalent dose or a toxicity adjusted dose. Overall cabazitaxel is more efficacious than docetaxel at both mid doses, on a dose equivalent basis.

### EXAMPLE 5

### Antitumor Activity of Cabazitaxel and Docetaxel Against Human Osteosarcoma DM113 in Nude Female Mice

In this second example, the better antitumor activity of cabazitaxel as compared to docetaxel for tumor growth inhibition was demonstrated in vivo.

The selected tumor model, DM113, was a low passage patient-derived tumor xenograft derived from an osteosarcoma taken from the lung of a 3 year old female patient.

The results of the experiments are reported below in Tables 13, 14 & 15 and in FIG. 8.

The tumor doubling time (in days; Td) was 7.9 days.

The following end points were used:
Toxicity was declared at dosages inducing ≧20% body weight loss or ≧10% drug death;
Antitumor efficacy was determined by calculating the ΔT/ΔC value in percent on day 28 post treatment initiation, according to the above mentioned formula;
Individual tumor volume changes from baseline were analyzed by a non-parametric two-way ANOVA-TYPE (with factors: group and repeated day from 3 to 28) followed by a post-hoc contrasts analysis, with Bonferroni-Holm adjustment for multiplicity, comparing all treated groups to the control group on day 28. Additionally, a non parametric two-way ANOVA-TYPE (with factors: treated group and repeated day from 3 to 46) was performed and followed by a contrast analysis, with Bonferroni-Holm adjustment for multiplicity, to compare at each day the effects of docetaxel and cabazitaxel when administered at the same doses.
At study completion, tumor growth delay (T-C) in days is calculated using the median time to endpoint (MTTE) value for each treatment (T) group versus control (C). The volume endpoint for T-C calculations was chosen to be 1600 mm³. A Log Rank multiple comparison test with Bonferroni-Holm adjustment for multiplicity was applied on individual TTE to compare the treated groups to the control group.
Tumor regressions (as explained above).

Results:

Treatment with cabazitaxel and docetaxel had minor impacts for the health status of the animals though weight losses were noted at the higher doses of 24.2 (11% versus 13%, respectively) and 15 mg/kg (9% and 8%, respectively) (Table 13).

Both Cabazitaxel and docetaxel demonstrate anti-tumor effects compared to the control via either tumor volume changes from baseline or T-C analysis (p<0.05 is for both end-points), except at the 5.8 mg/kg dose level of docetaxel (ΔT/ΔC=42.9%, p=0.3938; T-C=9 days, p=0.1771) (FIG. 8 and Table 14).

As shown in Table 15, comparison of tumor volume changes from baseline at equivalent dose levels demonstrated significantly greater activity for cabazitaxel compared to docetaxel at 9.3 mg/kg (on days 14 to 38), 15 mg/kg (on days 11 to 46), and 24.2 mg/kg (on days 11, 24 and 31 to 46).

Additionally, as reported in Table 14, when comparing the numbers of PR between treatment groups, a greater activity of cabazitaxel compared to docetaxel has been observed at 15 mg/kg (4/10 PR versus 0/10 PR, respectively) and at 24.2 mg/kg (5/10 PR versus 1/10 PR, respectively).

TABLE 13
Cabazitaxel and docetaxel toxicity in nude mice bearing
DM113 osteosarcoma

| | Dose | | Weight Nadir | | Drug Deaths | |
|---|---|---|---|---|---|---|
| Treatment | (mg/kg) | Route/Schedule | % | Day | Total | Day (#) |
| Control | — | i.v./q4dx3 | −1% | 3 | — | — |
| Cabazitaxel | 5.8 | i.v./q4dx3 | — | — | 0 | — |
| | 9.3 | i.v./q4dx3 | −3% | 3 | 0 | — |
| | 15 | i.v./q4dx3 | −9% | 14 | 0 | — |
| | 24.2 | i.v./q4dx3 | −11% | 11 | 0 | — |
| Docetaxel | 5.8 | i.v./q4dx3 | −2% | 3 | 0 | — |
| | 9.3 | i.v./q4dx3 | −3% | 17 | 0* | — |
| | 15 | i.v./q4dx3 | −8% | 17 | 0 | — |
| | 24.2 | i.v./q4dx3 | −13% | 17 | 0 | — |

*one animal died on day 35 with no known cause of death following necropsy

TABLE 14
Cabazitaxel and docetaxel antitumor activity in nude mice bearing DM113 osteosarcoma

| | | | Tumor Volume Data | | | | | | | |
|---|---|---|---|---|---|---|---|---|---|---|
| | | | (Day 28) | | | | | | | |
| | Dose | Route/ | Median | | | MTTE | | T-C | | |
| Treatment | (mg/kg) | Schedule | (mm³) | ΔT/ΔC % | pvalue* | (days) | pvalue** | (days) | n | #PR/CR/TFS |
| Control | — | i.v./q4dx3 | 1258 | | | 31 | — | — | 10 | — |
| Cabazitaxel | 5.8 | i.v./q4dx3 | 512.5 | 29.4 | p = 0.0442 | 47 | p = 0.0206 | 16 | 10 | 0/0/0 |
| | 9.3 | i.v./q4dx3 | 204 | 1.8 | p < .0001 | >59 | p = 0.0003 | >28 | 10 | 0/0/0 |
| | 15 | i.v./q4dx3 | 131 | −4.4 | p < .0001 | >59 | p < .0001 | >28 | 10 | 4/0/0 |
| | 24.2 | i.v./q4dx3 | 112 | −3.6 | p < .0001 | >59 | p < .0001 | >28 | 10 | 5/0/0 |
| Docetaxel | 5.8 | i.v./q4dx3 | 598 | 42.9 | p = 0.3938 | 41 | p = 0.1771 | 9 | 10 | 0/0/0 |
| | 9.3 | i.v./q4dx3 | 442 | 27.4 | p = 0.0235 | 49 | p = 0.0206 | 17 | 9 | 1/0/0 |
| | 15 | i.v./q4dx3 | 178 | 3.2 | p < .0001 | >59 | p < .0001 | >28 | 10 | 0/0/0 |
| | 24.2 | i.v./q4dx3 | 131 | 0 | p < .0001 | >59 | p < .0001 | >28 | 10 | 1/0/0 |

*Contrasts analysis versus control with Bonferroni-Holm adjustment for multiplicity following a non parametric two-way Anova-Type on tumor volume changes from baseline
*Log-Rank multiple comparisons test versus control on individuals time to event

TABLE 15
Comparison of the tumor volumes of the groups treated with cabazitaxel and
docetaxel at the same equi-toxic doses in nude mice bearing DM113 osteosarcoma

| | Median +/− MAD (number of subject) and pvalue* | | | |
|---|---|---|---|---|
| | Cabazitaxel | Docetaxel | Cabazitaxel | Docetaxel |
| | 5.8 mg/kg | 5.8 mg/kg | 9.3 mg/kg | 9.3 mg/kg |
| DAY 3 | 58 +/− 21.5 | 22.5 +/− 22.5 | 29.5 +/− 27 | 56.5 +/− 26.5 |
| | (n = 10) | (n = 10) | (n = 10) | (n = 10) |
| comparison | p = 1.0000 | | p = 1.0000 | |
| DAY 8 | 63.5 +/− 37 | 95 +/− 22.5 | 29.5 +/− 29.5 | 85.5 +/− 27 |
| | (n = 10) | (n = 10) | (n = 10) | (n = 10) |
| comparison | p = 0.5005 | | p = 0.3795 | |
| DAY 11 | 63.5 +/− 21.5 | 117.5 +/− 45 | 29.5 +/− 29.5 | 73 +/− 42.5 |
| | (n = 10) | (n = 10) | (n = 10) | (n = 10) |
| comparison | p = 0.3121 | | p = 0.3121 | |
| DAY 14 | 95 +/− 41.5 | 189 +/− 60.5 | 9 +/− 9 | 85.5 +/− 34.5 |
| | (n = 10) | (n = 10) | (n = 10) | (n = 10) |
| comparison | p = 0.1492 | | p = 0.0253 | |
| DAY 17 | 95 +/− 59 | 242.5 +/− 110 | 0 +/− 0 | 117.5 +/− 51.5 |
| | (n = 10) | (n = 10) | (n = 10) | (n = 10) |
| comparison | p = 0.1026 | | p = 0.0057 | |
| DAY 21 | 158.5 +/− 39.5 | 278.5 +/− 142 | 0 +/− 9 | 140.5 +/− 52 |
| | (n = 10) | (n = 10) | (n = 10) | (n = 10) |
| comparison | p = 0.1026 | | p = 0.0005 | |
| DAY 24 | 234 +/− 83.5 | 435 +/− 159 | 0 +/− 31 | 239 +/− 94.5 |
| | (n = 10) | (n = 10) | (n = 10) | (n = 10) |
| comparison | p = 0.1050 | | p = 0.0001 | |
| DAY 28 | 334.5 +/− 96 | 487 +/− 231.5 | 20 +/− 39.5 | 311 +/− 75.5 |
| | (n = 10) | (n = 10) | (n = 10) | (n = 10) |
| comparison | p = 0.1591 | | p = 0.0002 | |
| DAY 31 | 459.5 +/− 123 | 598 +/− 204 | 69 +/− 69 | 390 +/− 90.5 |
| | (n = 10) | (n = 9) | (n = 10) | (n = 10) |
| comparison | p = 0.2301 | | p = 0.0011 | |
| DAY 35 | 579.5 +/− 228 | 813 +/− 367 | 87.5 +/− 70.5 | 545 +/− 52 |
| | (n = 10) | (n = 9) | (n = 10) | (n = 9) |
| comparison | p = 0.2378 | | p = 0.0132 | |
| DAY 38 | 834.5 +/− 245 | 960 +/− 388 | 182 +/− 155.5 | 677 +/− 118 |
| | (n = 10) | (n = 8) | (n = 10) | (n = 9) |
| comparison | p = 0.3251 | | p = 0.0401 | |
| DAY 42 | 1097 +/− 248 | 1032 +/− 326 | 311 +/− 248 | 827.5 +/− 293.5 |
| | (n = 10) | (n = 7) | (n = 10) | (n = 8) |
| comparison | p = 0.4213 | | p = 0.2684 | |
| DAY 46 | 1548.5 +/− 438.5 | 1340 +/− 384 | 576 +/− 317.5 | 1187 +/− 347.5 |
| | (n = 10) | (n = 6) | (n = 10) | (n = 8) |

| | | |
|---|---|---|
| comparison | p = 0.6530 | p = 0.6530 |

| | Median +/− MAD (number of subject) and pvalue* | | | |
|---|---|---|---|---|
| | Cabazitaxel | Docetaxel | Cabazitaxel | Docetaxel |
| | 15 mg/kg | 15 mg/kg | 24.2 mg/kg | 24.2 mg/kg |
| DAY 3 | 13 +/− 13 | 26 +/− 26 | 9.5 +/− 9.5 | 29.5 +/− 13.5 |
| | (n = 10) | (n = 10) | (n = 10) | (n = 10) |
| comparison | p = 1.0000 | | p = 1.0000 | |
| DAY 8 | 0 +/− 0 | 26 +/− 26 | 0 +/− 26.5 | 22.5 +/− 22.5 |
| | (n = 10) | (n = 10) | (n = 10) | (n = 10) |
| comparison | p = 0.5005 | | p = 0.1597 | |
| DAY 11 | −23.5 +/− 23.5 | 13 +/− 16.5 | −40.5 +/− 34 | 0 +/− 22 |
| | (n = 10) | (n = 10) | (n = 10) | (n = 10) |
| comparison | p = 0.0056 | | p = 0.0240 | |
| DAY 14 | −40.5 +/− 30 | 13 +/− 16.5 | −43.5 +/− 28.5 | −9.5 +/− 35 |
| | (n = 10) | (n = 10) | (n = 10) | (n = 10) |
| comparison | p = 0.0011 | | p = 0.1105 | |
| DAY 17 | −40.5 +/− 30 | 13 +/− 16.5 | −40.5 +/− 24.5 | −9.5 +/− 26 |
| | (n = 10) | (n = 10) | (n = 10) | (n = 10) |
| comparison | p = 0.0011 | | p = 0.1026 | |
| DAY 21 | −40.5 +/− 30 | 0 +/− 19 | −40.5 +/− 24.5 | −9.5 +/− 26 |
| | (n = 10) | (n = 10) | (n = 10) | (n = 10) |
| comparison | p = 0.0129 | | p = 0.1026 | |
| DAY 24 | −50 +/− 36.5 | 22.5 +/− 23.5 | −40.5 +/− 40.5 | 0 +/− 0 |
| | (n = 10) | (n = 10) | (n = 10) | (n = 10) |
| comparison | p = 0.0018 | | p = 0.0282 | |
| DAY 28 | −50 +/− 36.5 | 36.5 +/− 35.5 | −40.5 +/− 40.5 | 0 +/− 9.5 |
| | (n = 10) | (n = 10) | (n = 10) | (n = 10) |
| comparison | p = 0.0008 | | p = 0.0704 | |
| DAY 31 | −53 +/− 36.5 | 42.5 +/− 29.5 | −40.5 +/− 34 | 0 +/− 9.5 |
| | (n = 10) | (n = 10) | (n = 10) | (n = 10) |
| comparison | p = 0.0004 | | p = 0.0332 | |
| DAY 35 | −59.5 +/− 29 | 61 +/− 61 | −51 +/− 41.5 | 0 +/− 9.5 |
| | (n = 10) | (n = 10) | (n = 10) | (n = 10) |
| comparison | p < 0.0001 | | p = 0.0118 | |
| DAY 38 | −59.5 +/− 39.5 | 61 +/− 81 | −51 +/− 41.5 | 0 +/− 9.5 |
| | (n = 10) | (n = 10) | (n = 10) | (n = 10) |
| comparison | p < 0.0001 | | p = 0.0089 | |
| DAY 42 | −59.5 +/− 39.5 | 107 +/− 90 | −51 +/− 41.5 | 0 +/− 9.5 |
| | (n = 10) | (n = 10) | (n = 10) | (n = 10) |
| comparison | p < 0.0001 | | p = 0.0057 | |
| DAY 46 | −59.5 +/− 42.5 | 155 +/− 153 | −51 +/− 41.5 | 50.5 +/− 47.5 |
| | (n = 10) | (n = 10) | (n = 10) | (n = 10) |

| | | |
|---|---|---|
| comparison | p < 0.0001 | p < 0.0001 |

*Contrasts analysis with Bonferroni-Holm adjustment for multiplicity following a two-way ANOVA-TYPE on tumor volume changes from baseline to compare, at each day, the groups treated with Cabazitaxel or Docetaxel at the same dose or at equi-toxic doses.

Conclusion:

These results demonstrate that both cabazitaxel and docetaxel demonstrate robust anti-tumor activity in this model. Furthermore, cabazitaxel demonstrates higher efficacy than docetaxel at the 9.3, 15, and 24.2 mg/kg dose levels.

### EXAMPLE 6

### Antitumor Activity of Cabazitaxel and Docetaxel Against Human Ewing'S Sarcoma DM101 in Nude Female Mice

In this third example, the better antitumor activity of cabazitaxel as compared to docetaxel for tumor growth inhibition was demonstrated in vivo.

The selected tumor model, DM101, was a low passage patient-derived tumor xenograft derived from an Ewing's sarcoma taken from the bone of a 17 year old male patient.

The results of the experiments are reported below in Tables 16, 17 & 18 and in FIG. 9.

The tumor doubling time (in days; Td) was 4 days.

The following end points were used:
Toxicity was declared at dosages inducing ≧20% body weight loss or ≧10% drug death;
Antitumor efficacy was determined by calculating the ΔT/ΔC value in percent on day 11 post treatment initiation, according to the above mentioned formula;
Individual tumor volume changes from baseline were analyzed by a non-parametric two-way ANOVA-TYPE (with factors: group and repeated day from 4 to 14) followed by a post-hoc contrasts analysis, with Bonferroni-Holm adjustment for multiplicity, comparing all treated groups to the control group on day 11. Additionally, a non parametric two-way ANOVA-TYPE (with factors: treated group and repeated day from 4 to 32) was performed and followed by a contrast analysis, with Bonferroni-Holm adjustment for multiplicity, to compare at each day the effects of docetaxel and cabazitaxel when administered at the same doses or at equi-toxic doses.
At study completion, tumor growth delay (T-C) in days is calculated using the median time to endpoint (MTTE) value for each T group versus C. The volume endpoint for T-C calculations was chosen to be 2000 mm³. A Log Rank multiple comparison test with Bonferroni-Holm adjustment for multiplicity was applied on individual TTE to compare the treated groups to the control group.
Tumor regressions (as explained above).

Results:

Both cabazitaxel and docetaxel demonstrate significant anti-tumor effects compared to the control via ΔT/ΔC on day 11 (FIG. 6 and Table 17).

Using weight loss as a gross indicator of toxicity (Table 16), docetaxel is more toxic than cabazitaxel at 24.2 mg/kg (17% versus 5% body weight loss).

At equivalent dose levels, the comparison of tumor volume changes from baseline shows no significant difference between the groups treated with cabazitaxel or docetaxel at dose 5.8 and 9.3 mg/kg. However, as shown in Table 18, starting from day 7, the groups treated with cabazitaxel at the 15 or 24.2 mg/kg doses were significantly different from the groups treated with docetaxel at the same dose (15 or 24.2 mg/kg, respectively) or at the equi-toxic dose (9.3 or 15 mg/kg, respectively).

In addition, animals treated with 15 or 24.2 mg/kg of cabazitaxel induced more CR and TFS as compared to docetaxel (9/9 CR and 7/9 TFS for cabazitaxel versus 4/9 CR and 1/9 TFS for docetaxel at 15 mg/kg; 9/9 CR and 8/9 TFS for cabazitaxel versus 3/9 CR and 2/9 TFS for docetaxel at 24.2 mg/kg).

TABLE 16
Cabazitaxel and docetaxel toxicity in nude mice bearing
DM101 Ewing's sarcoma

| | | | Weight | | Drug Deaths | |
|---|---|---|---|---|---|---|
| | Dose | | Nadir | | | Day |
| Treatment | (mg/kg) | Route/Schedule | % | Day | Total | (#) |
| Control | — | i.v./q4d × 3 | — | — | — | — |
| Cabazitaxel | 5.8 | i.v./q4d × 3 | — | — | 0 | — |
| | 9.3 | i.v./q4d × 3 | −2% | 7 | 0 | — |
| | 15 | i.v./q4d × 3 | −3% | 7 | 0 | — |
| | 24.2 | i.v./q4d × 3 | −5% | 11 | 0 | — |
| Docetaxel | 5.8 | i.v./q4d × 3 | −1% | 4 | 0 | — |
| | 9.3 | i.v./q4d × 3 | −4% | 7 | 0 | — |
| | 15 | i.v./q4d × 3 | −6% | 14 | 0 | — |
| | 24.2 | i.v./q4d × 3 | −17% | 14 | 0 | — |

TABLE 17
Cabazitaxel and docetaxel antitumor activity in nude mice bearing DM101 Ewing's sarcoma

| | | | Tumor Volume Data (Day 11) | | | | | | | |
|---|---|---|---|---|---|---|---|---|---|---|
| | Dose | | Median | | | MTTE | | T-C | | |
| Treatment | (mg/kg) | Route/Schedule | (mm³) | ΔT/ΔC % | pvalue* | (days) | pvalue** | (days) | n | #PR/CR/TFS |
| Control | — | i.v./q4d × 3 | 940 | | | 16.9 | — | — | 10 | — |
| Cabazitaxel | 5.8 | i.v./q4d × 3 | 204 | 8.9 | p = 0.0044 | 34.8 | p = 0.0576 | 17.9 | 9 | 0/0/0 |
| | 9.3 | i.v./q4d × 3 | 255 | 9.4 | p = 0.0004 | 23.9 | p = 0.1185 | 7 | 9 | 1/1/1 |
| | 15 | i.v./q4d × 3 | 0 | −16 | p < 0.0001 | >61 | p = 0.0002 | >44.1 | 9 | 9/9/7 |
| | 24.2 | i.v./q4d × 3 | 0 | −18.3 | p < 0.0001 | >61 | p < 0.0001 | >44.1 | 9 | 9/9/8 |
| Docetaxel | 5.8 | i.v./q4d × 3 | 366 | 24.7 | p = 0.0397 | 35 | p = 0.1185 | 18.1 | 9 | 0/0/0 |
| | 9.3 | i.v./q4d × 3 | 505 | 36.8 | p = 0.0004 | 30.5 | p = 0.0576 | 13.6 | 9 | 4/4/1 |
| | 15 | i.v./q4d × 3 | 204 | 8.9 | p = 0.0002 | 50.9 | p = 0.0562 | 34 | 9 | 4/4/1 |
| | 24.2 | i.v./q4d × 3 | 300 | 14.9 | p = 0.0001 | 32.3 | p = 0.0576 | 15.4 | 9 | 4/3/2 |

*Contrasts analysis versus control with Bonferroni-Holm adjustment for multiplicity following a non parametric two-way Anova-Type on tumor volume changes from baseline
**Log-Rank multiple comparisons test versus control on individual time to event

TABLE 18
Comparison of the tumor volumes of cabazitaxel and docetaxel at the same equi-toxic doses in nude mice
bearing DM101 Ewing's sarcoma

| | Median +/− MAD (number of subject) and pvalue* | | | | | | | |
|---|---|---|---|---|---|---|---|---|
| | Cabazitaxel | Cabazitaxel | Cabazitaxel | Cabazitaxel | Docetaxel | | Docetaxel | Docetaxel |
| | 5.8 mg/kg | 9.3 mg/kg | 15 mg/kg | 24.2 mg/kg | 5.8 mg/kg | Docetaxel 9.3 mg/kg | 15 mg/kg | 24.2 mg/kg |
| DAY 4 | 0 +/− 0 (n = 9) | 0 +/− 33 | 0 +/− 0 (n = 9) | −19 +/− 19 | 51 +/− 38 | 25 +/− 25 | 26 +/− 25 | 26 +/− 40 |
| | | (n = 9) | | (n = 9) | (n = 9) | (n = 9) | (n = 9) | (n = 9) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 1.0000 | p = 1.0000 | p = 1.0000 | p = 1.0000 | p = 1.0000 | p = 1.0000 | |
| DAY 7 | 34 +/− 34 | 13 +/− 41 | −113 +/− 32 | −150 +/− 52 | 98 +/− 79 | 96 +/− 172 | 73 +/− 109 | 122 +/− |
| | (n = 9) | (n = 9) | (n = 9) | (n = 9) | (n = 9) | (n = 9) | (n = 9) | 184 (n = 9) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 1.0000 | p = 0.0159 | p = 0.0042 | p = 0.6555 | p = 0.0174 | p = 0.0043 | |
| DAY 11 | 73 +/− 73 | 77 +/− 118 | −131 +/− 47 | −150 +/− 38 | 202 +/− 183 | 301 +/− 223 | 73 +/− 186 | 122 +/− |
| | (n = 9) | (n = 9) | (n = 9) | (n = 9) | (n = 9) | (n = 9) | (n = 9) | 184 (n = 9) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 1.0000 | p = 0.0019 | p = 0.0019 | p = 0.5726 | p = 0.0015 | p = 0.0019 | |
| DAY 14 | 155 +/− 136 | 188 +/− 241 | −131 +/− 47 | −150 +/− 38 | 446 +/− 393 | 472 +/− 550 | 73 +/− 251 | 122 +/− |
| | (n = 9) | (n = 9) | (n = 9) | (n = 9) | (n = 9) | (n = 9) | (n = 9) | 215 (n = 9) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 1.0000 | p = 0.0008 | p = 0.0012 | p = 0.4725 | p = 0.0003 | p = 0.0008 | |
| DAY 17 | 306 +/− 152 | 498 +/− 485 | −131 +/− 47 | −150 +/− 38 | 640 +/− 621 | 750 +/− 828 | 169 +/− 347 | 122 +/− |
| | (n = 9) | (n = 9) | (n = 9) | (n = 9) | (n = 9) | (n = 9) | (n = 9) | 234 (n = 9) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 1.0000 | p = 0.0002 | p = 0.0003 | p = 0.6650 | p < 0.0001 | p = 0.0002 | |
| DAY 20 | 489 +/− 199 | 766 +/− 713 | −131 +/− 47 | −150 +/− 38 | 813 +/− 756 | 813 +/− 891 | 290 +/− 468 | 394 +/− |
| | (n = 9) | (n = 9) | (n = 9) | (n = 9) | (n = 9) | (n = 9) | (n = 9) | 407 (n = 9) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 1.0000 | p = 1.0000 | p < 0.0001 | p < 0.0001 | p = 0.7250 | p < 0.0001 | p < 0.0001 | |
| DAY 25 | 696.5 +/− | 1095 +/− | −131 +/− 47 | −150 +/− 38 | 766 +/− 478 | −78 +/− 110 | −78 +/− 100 | 351.5 +/− |
| | 295.5 (n = 8) | 1023 (n = 9) | (n = 9) | (n = 9) | (n = 7) | (n = 7) | (n = 7) | 463.5 |
| | | | | | | | | (n = 8) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 0.9784 | p = 0.6388 | p = 0.0001 | p < 0.0001 | p = 0.7289 | p < 0.0001 | p = 0.0001 | |
| DAY 28 | 1097 +/− 117 | 182.5 +/− 248 | −131 +/− 47 | −150 +/− 38 | 1140 +/− 564 | −78 +/− 36 | −84.5 +/− 70 | 681 +/− |
| | (n = 8) | (n = 6) | (n = 9) | (n = 9) | (n = 7) | (n = 6) | (n = 6) | 793 (n = 8) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 0.9886 | p = 0.5325 | p = 0.0003 | p < 0.0001 | p = 0.5169 | p < 0.0001 | p = 0.0003 | |
| DAY 32 | 1396 +/− | 385 +/− 414 | −131 +/− 40 | −150 +/− 38 | 1694 +/− 281 | −78 +/− 34 | −91 +/− 40 | 254 +/− |
| | 182.5 (n = 8) | (n = 6) | (n = 9) | (n = 9) | (n = 7) | (n = 5) | (n = 5) | 366 (n = 5) |
| Comparison | Docetaxel | Docetaxel | Docetaxel | Docetaxel | Cabazitaxel | Cabazitaxel | Cabazitaxel | |
| versus | 5.8 mg/kg | 9.3 mg/kg | 15 mg/kg | 24.2 mg/kg | 9.3 mg/kg | 15 mg/kg | 24.2 mg/kg | |
| | p = 0.8900 | p = 0.5900 | p = 0.0018 | p < 0.0001 | p = 0.5900 | p < 0.0001 | p = 0.0016 | |

*Contrasts analysis with Bonferroni-Holm adjustment for multiplicity following a two-way ANOVA-TYPE on tumor volume changes from baseline to compare, at each day, the groups treated with Cabazitaxel or Docetaxel at the same dose or at equi-toxic doses.

Conclusion: Both cabazitaxel and docetaxel demonstrate robust anti-tumor activity in this model. Cabazitaxel at the 15 or 24.2 mg/kg doses was significantly more active than docetaxel at the same dose (15 or 24.2 mg/kg, respectively) or at the equi-toxic dose (9.3 or 15 mg/kg, respectively).

What is claimed is:

1. The compound of formula (I): 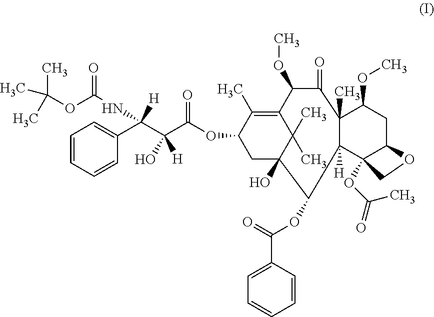 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]
which may be in the form of an anhydrous base, a hydrate or a solvate,
for its use for the treatment of pediatric cancers.

2. The compound for the use of claim 1, for the treatment of pediatric solid tumors.

3. The compound for the use of claim 2, wherein the pediatric solid tumors are chosen from the group consisting of: anaplastic astrocytomas, glioblastomas, anaplastic oligodendrogliomas, oligoastrocytomas, anaplastic ependymomas, nephroblastoma, medulloblastomas, neuroblastomas, Wilm's tumors, rhabdomyosarcomas, chondrosarcomas, Ewing's sarcomas and osteosarcomas.

4. The compound for the use of claim 1, for the treatment of rhabdomyosarcoma.

5. The compound for the use of claim 1, for the treatment of Ewing's tumor.

6. The compound for the use of claim 1, for the treatment of osteosarcoma.

7. The compound for the use of claim 1, for the treatment of high grade gliomas.

8. The compound for the use of claim 1, wherein said compound is in the form of an acetone solvate.

9. The compound for the use of claim 8, wherein the acetone solvate comprises from 5% to 8% by weight of acetone.

10. The compound for the use of claim 1, wherein said compound is administered by parenteral route.

11. The compound for the use of claim 10, wherein said compound is administered by intravenous route.
